(19) United States
(12) Patent Application Publication
(10) Pub. No.: US 2021/0171604 A1
(43) Pub. Date: Jun. 10, 2021

(54) PEPTIDES FOR THE TREATMENT OF TYPE 2 DIABETES

(71) Applicant: YISSUM RESEARCH DEVELOPMENT COMPANY OF THE HEBREW UNIVERSITY OF JERUSALEM LTD., Jerusalem (IL)

(72) Inventor: Shmuel BEN-SASSON, Jerusalem (IL)

(21) Appl. No.: 16/619,642

(22) PCT Filed: Jun. 10, 2018
(86) PCT No.: PCT/IL2018/050632

§ 371 Date: Dec. 5, 2019

Related U.S. Application Data
(60) Provisional application No. 62/517,924, filed on Jun. 11, 2017.

(51) Int. Cl. C07K 14/705; C12N 9/02; A61K 47/62; A61P 3/10

(52) U.S. Cl. CPC C07K 14/70567; C12Y 114/99003; A61K 38/00; A61K 47/62; A61P 3/10; C12N 9/0083

(57) ABSTRACT

Short peptides and peptidomimetics useful for treating Type 2 diabetes are provided, and methods for treating and/or preventing Type 2 diabetes and related conditions.

### FIELD OF THE INVENTION

The present invention relates to short peptides effective in preventing and/or treating Type 2 diabetes and related conditions such as metabolic syndrome.

### BACKGROUND OF THE INVENTION

Type 2 diabetes (T2D), once known as non-insulin-dependent diabetes (NIDDM), is a metabolic disorder characterized by hyperglycemia (high blood sugar) caused by inability of the body cells to use insulin efficiently. The latter is known as insulin resistance. In insulin resistance, body cells such as muscle, fat, and liver cells do not respond properly to insulin and as a result increasing levels of insulin are needed in order to facilitate glucose uptake by the cells. As long as insulin over-production by the pancreas is sufficient to overcome the insulin resistance, blood glucose levels typically stay within the normal healthy range. However, over time, sufficiently high insulin levels can no longer be produced and glucose levels in the blood rise above the normal range, leading to the development of T2D.

T2D is thought to result from a combination of genetic and environmental factors (Kato et al., 2013, J Diabetes Investig. 4:233-44). The risk of developing T2D is greatly increased when associated with lifestyle factors such as high blood pressure, overweight or obesity, insufficient physical activity, poor diet and an ‘apple shape’ body where extra weight is carried around the waist. T2D can often initially be managed with healthy eating and regular physical activity. However, over time most people with T2D will also need medications and insulin.

T2D is associated with a number of co-morbidities and complications, including hypertension, dyslipidemia, cardiovascular diseases, blindness and eye problems, and increased risk of heart attacks, strokes, damage to kidneys and limb amputations.

Despite extensive research in the field, there is no cure for T2D, and currently available treatments are mainly symptomatic.

Heme oxygenase (HO) is the rate-limiting enzyme in the catabolism of the cofactor heme in cells, a process that leads to formation of the bile pigment biliverdin, free iron, and carbon monoxide (CO). Biliverdin formed in this reaction is rapidly converted to bilirubin. HO is known to exist as two isoenzymes, termed HO-1 and HO-2. HO-1 is an enzyme inducible by its substrate heme and also in response to various stress conditions, including acute starvation (fasting), oxidative stress, hypoxia, heavy metals, cytokines, etc. (Abraham et al., 2008, Pharmacol. Rev., 60: 79-127). The substrate heme was found as well to be elevated during fasting (Handschin et al., 2005, Cell, 122: 505-15). HO-2 is a constitutive isoform that is expressed under homeostatic conditions.

Increased levels of HO-1 were found in T2D. Recent work has shown that overexpression of heme oxygenase-1 (HO-1) is associated with increased risk of developing metabolic syndrome, insulin resistance and T2D (Jais et al., 2014, Cell 158: 25-40). Jais et al., have suggested HO-1 inhibition as a potential therapeutic strategy for metabolic disease.

Hypoxia-inducible factor 1-alpha (HIF1α) is a subunit of the heterodimeric transcription factor hypoxia-inducible factor 1 (HIF-1) protein, which is considered as the master transcriptional regulator of cellular and developmental response to hypoxia.

U.S. Pat. No. 8,143,228 discloses inter alia methods and pharmaceutical compositions for the treatment of cancer or acute ischemia. Among others, HIF-1 alpha derived peptides or peptide analogs are disclosed.

There is a medical need for improved compositions and methods for treating Type 2 diabetes and associated conditions.

### SUMMARY OF THE INVENTION

The present invention provides according to some aspects short peptides and peptidomimetics useful for treating Type 2 diabetes, and methods for treating Type 2 diabetes and related conditions.

The present invention is based in part on the unexpected finding that peptides derived from a particular segment of the human protein heme oxygenase-1 (HO-1) or from sequences in human hypoxia-inducible factor 1 alpha (HIF1α) and DQX1 that have homology to the HO-1 sequence, are effective in inhibiting insulin-resistance in normal fasting mice and lowering blood glucose levels in obese diabetic mice.

Without wishing to be bound by any particular theory of mechanism of action, it is contemplated that the peptides disclosed herein competitively inhibit a post-translational modification of HO-1 that renders HO-1 inactive, thereby maintaining HO-1 in an active form. It is contemplated that maintaining HO-1 in an active form prevents accumulation of heme and subsequent downstream processes leading to development of insulin resistance and Type 2 diabetes.

According to one aspect, the present invention provides a synthetic or recombinant peptide or peptidomimetic of 7-20 amino acids, the peptide comprising the sequence X₁-X₂-X₃-X₄-Lys-Gly-Gln-X₅-Thr-X₆-X₇ (SEQ ID NO: 1), wherein:

X₁ is an amino acid residue other than Met and His;

X₂ is absent or represents a stretch of two amino acid residues selected from the group consisting of: Arg-X₈, wherein X₈ is any amino acid residue, Asp-Met and Leu-Gln;

X₃ is selected from the group consisting of Phe, Tyr, His, Leu, Ala, Ser and NorVal;

X₄ is selected from Thr and Gln;

X₅ is selected from Val and Ser;

X₆ is absent or selected from the group consisting of a positively charged amino acid residue, Ser and Val; and

X₇ is absent or represents a positively charged amino acid residue, optionally conjugated with a label. In some embodiments, the label is a detectable label.

In some embodiments, the peptide or peptidomimetic comprises a sequence selected from the group consisting of:

(SEQ ID NO: 2)
X₁-Arg-Asn-Phe-Gln-Lys-Gly-Gln-Val-Thr-Arg;
(SEQ ID NO: 3)
X₁-Asp-Met-Phe-Thr-Lys-Gly-Gln-Val-Thr-Arg;
(SEQ ID NO: 4)
X₁-Leu-Gln-Ser-Thr-Lys-Gly-Gln-Ser-Thr-Ser;
(SEQ ID NO: 5)
X₁-Leu-Gln-Ser-Gln-Lys-Gly-Gln-Ser-Thr-Ser;
and
(SEQ ID NO: 6)
X₁-Arg-Gly-Phe-Gln-Lys-Gly-Gln-Val-Thr-Val,

wherein X₁ is as defined above.

In some embodiments, the peptide or peptidomimetic comprises the sequence X₁-X₂-X₃-X₄-Lys-Gly-Gln-Val-Thr-X₆-X₇ (SEQ ID NO: 7), wherein:

X₁ is an amino acid residue other than Met;

X₂ is absent or represents Arg-Asn;

X₃ is selected from the group consisting of Phe, Tyr, His, Leu, Ala, and NorVal;

X₄ is Thr or Gln;

X₆ is absent or represents a positively charged amino acid residue; and

X₇ is absent or represents a positively charged amino acid residue, optionally conjugated with a label.

In some embodiments, the peptide or peptidomimetic comprises the sequence Gly-X₂-X₃-X₄-Lys-Gly-Gln-Val-Thr-X₆-X₇ (SEQ ID NO: 8), wherein X₂ is absent or represents Arg-Asn; X₃ is selected from the group consisting of Phe, Tyr, His, Leu, Ala, and NorVal; X₄ is Thr or Gln; X₆ is absent or represents a positively charged amino acid residue; and X₇ is absent or represents a positively charged amino acid residue, optionally conjugated with a label.

In some embodiments, X₆ is Arg.

In some embodiments, X₇ is absent. In other embodiments, X₇ is a positively charged amino acid residue conjugated with a label. In some embodiments, X₇ is a modified Lys residue Lys(Z), wherein Z is the label connected to the epsilon amino group of the Lys residue. In some embodiments Z is biotin or a dansyl moiety.

In some embodiments, X₁-X₂-X₃-X₄ represent a stretch of amino acid residues selected from the group consisting of:

(SEQ ID NO: 9)
Gly-Arg-Asn-Phe-Gln;
(SEQ ID NO: 10)
Gly-Arg-Asn-His-Gln;
(SEQ ID NO: 11)
Gly-Arg-Asn-Leu-Gln;
(SEQ ID NO: 12)
Gly-Arg-Asn-NorVal-Gln;
(SEQ ID NO: 13)
Gly-Arg-Asn-Ala-Gln;
(SEQ ID NO: 14)
Gly-Arg-Asn-Tyr-Gln;
and
(SEQ ID NO: 15)
Gly-Phe-Thr.

In some embodiments, X₆-X₇ represent Arg or Arg-Lys(Z), wherein Z is a label connected to the epsilon amino group of the Lys residue. In some embodiments Z is biotin or a dansyl moiety.

In other embodiments, X₆ and X₇ are absent.

In some embodiments, the peptide comprises the sequence X₁-Arg-Asn-X₃-Gln-Lys-Gly-Gln-Val-Thr-Arg-X₇ (SEQ ID NO: 16), wherein X₁ is an amino acid residue other than Met; X₃ is selected from the group consisting of Phe, Tyr, His, Leu, Ala, and NorVal; and X₇ is absent or represents a positively charged amino acid residue, optionally conjugated with a label.

In some embodiments, the peptide comprises the sequence Gly-Arg-Asn-X₃-Gln-Lys-Gly-Gln-Val-Thr-Arg-X₇ (SEQ ID NO: 17) wherein X₃ is selected from the group consisting of Phe, Tyr, His, Leu, Ala, and NorVal; and X₇ is absent or represents a positively charged amino acid residue, optionally modified with a label.

In some embodiments, the peptide comprises a sequence selected from the group consisting of:

(SEQ ID NO: 18)
Gly-Arg-Asn-Phe-Gln-Lys-Gly-Gln-Val-Thr-Arg;
(SEQ ID NO: 19)
Gly-Arg-Asn-His-Gln-Lys-Gly-Gln-Val-Thr-Arg;
(SEQ ID NO: 20)
Gly-Arg-Asn-Leu-Gln-Lys-Gly-Gln-Val-Thr-Arg;
(SEQ ID NO: 21)
Gly-Arg-Asn-NorVal-Gln-Lys-Gly-Gln-Val-Thr-Arg;
(SEQ ID NO: 22)
Gly-Arg-Asn-Phe-Gln-Lys-Gly-Gln-Val-Thr-Arg-
Lys(Z);
(SEQ ID NO: 23)
Gly-Arg-Asn-Ala-Gln-Lys-Gly-Gln-Val-Thr-Arg;
(SEQ ID NO: 24)
Gly-Arg-Asn-Tyr-Gln-Lys-Gly-Gln-Val-Thr-Arg;
and
(SEQ ID NO: 25)
Gly-Arg-Asn-Tyr-Gln-Lys-Gly-Gln-Val-Thr-Arg-
Lys(Z),

wherein Z is as defined above.

In some embodiments, the peptide consists of a sequence selected from the group consisting of:

(SEQ ID NO: 18)
Gly-Arg-Asn-Phe-Gln-Lys-Gly-Gln-Val-Thr-Arg;
(SEQ ID NO: 19)
Gly-Arg-Asn-His-Gln-Lys-Gly-Gln-Val-Thr-Arg;
(SEQ ID NO: 20)
Gly-Arg-Asn-Leu-Gln-Lys-Gly-Gln-Val-Thr-Arg;
(SEQ ID NO: 21)
Gly-Arg-Asn-NorVal-Gln-Lys-Gly-Gln-Val-Thr-Arg;
(SEQ ID NO: 22)
Gly-Arg-Asn-Phe-Gln-Lys-Gly-Gln-Val-Thr-Arg-
Lys(Z);
(SEQ ID NO: 23)
Gly-Arg-Asn-Ala-Gln-Lys-Gly-Gln-Val-Thr-Arg;
(SEQ ID NO: 24)
Gly-Arg-Asn-Tyr-Gln-Lys-Gly-Gln-Val-Thr-Arg;
and
(SEQ ID NO: 25)
Gly-Arg-Asn-Tyr-Gln-Lys-Gly-Gln-Val-Thr-Arg-
Lys(Z),

wherein Z is as defined above.

In some embodiments, the peptide comprising the sequence X₁-Phe-Thr-Lys-Gly-Gln-Val-Thr (SEQ ID NO: 26), wherein X₁ is an amino acid residue other than Met.

In some embodiments, the peptide comprises the sequence Gly-Phe-Thr-Lys-Gly-Gln-Val-Thr (SEQ ID NO: 27).

In additional embodiments, the peptide consists of the sequence Gly-Phe-Thr-Lys-Gly-Gln-Val-Thr (SEQ ID NO: 27).

In some embodiments, the amino terminus of the peptide is modified with an amino-terminal blocking group selected from the group consisting of an acyl, alkyl and aryl. Each possibility represents a separate embodiment of the present invention.

In some embodiments, the carboxy terminus of the peptide is modified with a moiety selected from amide, ester and alcohol group. Each possibility represents a separate embodiment of the present invention.

According to a further aspect, the present invention provides a conjugate comprising the peptide or peptidomimetic of the invention and at least one moiety selected from the group consisting of a permeability-enhancing moiety, a detectable label and a carrier.

In some embodiments, the conjugate is according to Formula I:

R₁-X₁-X₂-X₃-X₄-Lys-Gly-Gln-X₅-Thr-X₆-X₇-R₂,

wherein R₁ is selected from the group consisting of a permeability-enhancing moiety and a detectable label, linked via a direct bond or via a linker; R₂ designates OH of an unmodified carboxy terminal group or a modified carboxyl terminal group; and X₁-X₇ are as defined above.

In some embodiments, R₁ is a permeability-enhancing moiety. In some embodiments, the permeability-enhancing moiety is a fatty acid residue.

In some embodiments, the fatty acid residue is a C12-C20 fatty acid.

In some embodiments, the fatty acid residue is selected from the group consisting of a myristoyl (Myr), a stearoyl (Stear) and a palmitoyl (Palm).

In some embodiments, the fatty acid residue is a myristoyl (Myr).

In some embodiments, the fatty acid residue is a stearoyl (Stear).

In some embodiments, the fatty acid residue is a palmitoyl (Palm).

In some embodiments, R₂ is a carboxyl group selected from amide, ester and alcohol group.

In some embodiments, R₂ is an amide group.

In some embodiments, the peptide consists of 7-15 amino acids.

In some embodiments, the conjugate is according to Formula Ia:

R₁-X₁-Arg-Asn-X₃-Gln-Lys-Gly-Gln-Val-Thr-Arg-X₇-R₂,

wherein R₁, R₂, X₁, X₃ and X₇ are as defined above.

In some embodiments, the conjugate is according to the following formula:

R₁-Gly-Arg-Asn-X₃-Gln-Lys-Gly-Gln-Val-Thr-Arg-X₇-R₂,

wherein R₁ and R₂ are as defined above, and wherein:

X₁ is an amino acid residue other than Met;

X₃ is selected from the group consisting of Phe, Tyr, His, Leu, Ala, and NorVal; and

X₇ is absent or represents a positively charged amino acid residue, optionally modified with a label.

In some embodiments, the conjugate is selected from the group consisting of:

R₁-Gly-Arg-Asn-Phe-Gln-Lys-Gly-Gln-Val-Thr-Arg-R₂;

R₁-Gly-Arg-Asn-His-Gln-Lys-Gly-Gln-Val-Thr-Arg-R₂;

R₁-Gly-Arg-Asn-Leu-Gln-Lys-Gly-Gln-Val-Thr-Arg-R₂;

R₁-Gly-Arg-Asn-NorVal-Gln-Lys-Gly-Gln-Val-Thr-Arg-R₂;

R₁-Gly-Arg-Asn-Phe-Gln-Lys-Gly-Gln-Val-Thr-Arg-Lys(Z)-R₂;

R₁-Gly-Arg-Asn-Ala-Gln-Lys-Gly-Gln-Val-Thr-Arg-R₂;

R₁-Gly-Arg-Asn-Tyr-Gln-Lys-Gly-Gln-Val-Thr-Arg-R₂; and

R₁-Gly-Arg-Asn-Tyr-Gln-Lys-Gly-Gln-Val-Thr-Arg-Lys(Z)-R₂,

wherein R₁, R₂, and Z are as defined above.

In some particular embodiments, the conjugate is selected from the group consisting of:

(SEQ ID NO: 28)
Myr-Gly-Arg-Asn-Phe-Gln-Lys-Gly-Gln-Val-Thr-Arg-
NH₂,;
(SEQ ID NO: 29)
Stear-Gly-Arg-Asn-Phe-Gln-Lys-Gly-Gln-Val-Thr-Arg-
NH₂,;
(SEQ ID NO: 30)
Palm-Gly-Arg-Asn-His-Gln-Lys-Gly-Gln-Val-Thr-Arg-
NH₂;
(SEQ ID NO: 31)
Stear-Gly-Arg-Asn-His-Gln-Lys-Gly-Gln-Val-Thr-Arg-
NH₂;
(SEQ ID NO: 32)
Myr-Gly-Arg-Asn-Leu-Gln-Lys-Gly-Gln-Val-Thr-Arg-
NH₂;
(SEQ ID NO: 33)
Myr-Gly-Arg-Asn-NorVal-Gln-Lys-Gly-Gln-Val-Thr-
Arg-NH₂;
(SEQ ID NO: 34)
Myr-Gly-Arg-Asn-Phe-Gln-Lys-Gly-Gln-Val-Thr-Arg-
Lys(Biotin)-NH₂;
(SEQ ID NO: 35)
Myr-Gly-Arg-Asn-Ala-Gln-Lys-Gly-Gln-Val-Thr-Arg-
NH₂;
(SEQ ID NO: 36)
Palm-Gly-Arg-Asn-Phe-Gln-Lys-Gly-Gln-Val-Thr-Arg-
NH₂,;
(SEQ ID NO: 37)
Stear-Gly-Arg-Asn-Tyr-Gln-Lys-Gly-Gln-Val-Thr-Arg-
NH₂;
and
(SEQ ID NO: 38)
Stear-Gly-Arg-Asn-Tyr-Gln-Lys-Gly-Gln-Val-Thr-Arg-
Lys(Dansyl)-NH₂.

In some embodiments, the conjugate is according to Formula Ib:

R₁-X₁-Phe-Thr-Lys-Gly-Gln-Val-Thr-R₂,

wherein R₁ and R₂ are as defined above, and wherein X₁ an amino acid residue other than Met.

In some embodiments, the conjugate is R₁-Gly-Phe-Thr-Lys-Gly-Gln-Val-Thr-R₂, wherein R₁ and R₂ are as defined above.

In some particular embodiments, the conjugate is Myr-Gly-Phe-Thr-Lys-Gly-Gln-Val-Thr-NH₂ (SEQ ID NO: 39).

According to another aspect, the present invention provides a pharmaceutical composition comprising as an active ingredient a peptide, peptidomimetic or conjugate of the invention and a pharmaceutically acceptable carrier.

The peptides, peptidomimetics and conjugates included in the pharmaceutical compositions of the invention are described above. According to some specific embodiments, the pharmaceutical composition comprises a peptide, peptidomimetic, or conjugate according to any one of formulae I, Ia, Ib and SEQ ID NOs: 1-39. Each possibility represents a separate embodiment of the present invention.

In some embodiments, the pharmaceutical composition is for use in the treatment of Type 2 diabetes.

According to yet another aspect, the present invention provides a method for treating Type 2 diabetes in a subject in need thereof, the method comprising administering to the subject a pharmaceutical composition comprising a peptide, peptidomimetic or conjugate of the present invention.

According to some specific embodiments, the method comprises administering a pharmaceutical composition comprising a peptide, peptidomimetic, or conjugate according to any one of formulae I, Ia, Ib and SEQ ID NOs: 1-39. Each possibility represents a separate embodiment of the present invention.

According to yet another aspect, the present invention provides a method for treating Type 2 diabetes in a subject in need thereof, the method comprising administering to the subject a pharmaceutical composition comprising a peptide or a peptidomimetic of 7-20 amino acids, the peptide or peptidomimetic comprising the sequence X₁-X₂-X₃-X₄-Lys-Gly-Gln-X₅-Thr-X₆-X₇ (SEQ ID NO: 40), wherein:

X₁ is any amino acid residue;

X₂ is absent or represents a stretch of two amino acid residues selected from the group consisting of: Arg-X₈, wherein X₈ is any amino acid residue, Asp-Met and Leu-Gln;

X₃ is selected from the group consisting of Phe, Tyr, His, Leu, Ala, Ser and NorVal;

X₄ is selected from Thr and Gln;

X₅ is selected from Val and Ser;

X₆ is absent or selected from the group consisting of a positively charged amino acid residue, Ser and Val; and

X₇ is absent or represents a positively charged amino acid residue, optionally conjugated with a label. In some embodiments, the label is a detectable label.

According to yet another aspect, the present invention provides a pharmaceutical composition for use in the treatment of Type 2 diabetes in a subject in need thereof, the pharmaceutical composition comprising a peptide or a peptidomimetic of 7-20 amino acids, the peptide or peptidomimetic comprising the sequence X₁-X₂-X₃-X₄-Lys-Gly-Gln-X₅-Thr-X₆-X₇ (SEQ ID NO: 40), wherein X₁ is any amino acid residue; X₂ is absent or represents a stretch of two amino acid residues selected from the group consisting of: Arg-X₈, wherein X₈ is any amino acid residue, Asp-Met and Leu-Gln; X₃ is selected from the group consisting of Phe, Tyr, His, Leu, Ala, Ser and NorVal; X₄ is selected from Thr and Gln; X₅ is selected from Val and Ser; X₆ is absent or selected from the group consisting of a positively charged amino acid residue, Ser and Val; and X₇ is absent, or represents a positively charged amino acid residue, optionally modified with a label.

In some embodiments, the peptide or peptidomimetic comprises a sequence selected from the group consisting of:

(SEQ ID NO: 2)
X₁-Arg-Asn-Phe-Gln-Lys-Gly-Gln-Val-Thr-Arg;
(SEQ ID NO: 3)
X₁-Asp-Met-Phe-Thr-Lys-Gly-Gln-Val-Thr-Arg;
(SEQ ID NO: 4)
X₁-Leu-Gln-Ser-Thr-Lys-Gly-Gln-Ser-Thr-Ser;
(SEQ ID NO: 5)
X₁-Leu-Gln-Ser-Gln-Lys-Gly-Gln-Ser-Thr-Ser;
and
(SEQ ID NO: 6)
X₁-Arg-Gly-Phe-Gln-Lys-Gly-Gln-Val-Thr-Val,

wherein X₁ is any amino acid residue.

In some embodiments, the peptide or peptidomimetic comprises the sequence Gly-X₂-X₃-X₄-Lys-Gly-Gln-Val-Thr-X₆-X₇ (SEQ ID NO: 8), wherein:

X₂ is absent or represents Arg-Asn;

X₃ is selected from the group consisting of Phe, Tyr, His, Leu, Ala, and NorVal;

X₄ is Thr or Gln;

X₆ is absent or represents a positively charged amino acid residue; and

X₇ is absent or represents a positively charged amino acid residue, optionally conjugated with a label.

In some embodiments, X₆ is Arg.

In some embodiments, X₇ is absent. In other embodiments, X₇ is a positively charged amino acid residue conjugated with a label. In some embodiments, X₇ is a modified Lys residue Lys(Z), wherein Z is the label connected to the epsilon amino group of the Lys residue. In some embodiments Z is biotin or a dansyl moiety.

In some embodiments, X₁-X₂-X₃-X₄ represent a stretch of amino acid residues selected from the group consisting of:

(SEQ ID NO: 9)
Gly-Arg-Asn-Phe-Gln;
(SEQ ID NO: 10)
Gly-Arg-Asn-His-Gln;
(SEQ ID NO: 11)
Gly-Arg-Asn-Leu-Gln;
(SEQ ID NO: 12)
Gly-Arg-Asn-NorVal-Gln;
(SEQ ID NO: 13)
Gly-Arg-Asn-Ala-Gln;
(SEQ ID NO: 14)
Gly-Arg-Asn-Tyr-Gln;
and
(SEQ ID NO: 15)
Gly-Phe-Thr.

In some embodiments, the peptide or peptidomimetic comprises the sequence X₁-Arg-Asn-X₃-Gln-Lys-Gly-Gln-Val-Thr-Arg-X₇ (SEQ ID NO: 16), wherein X₁ is any amino acid residue; X₃ is selected from the group consisting of Phe, Tyr, His, Leu, Ala, Ser and NorVal; and X₇ is absent, or represents a positively charged amino acid residue, optionally conjugated with a label.

In some embodiments, the peptide comprises the sequence Gly-Arg-Asn-X₃-Gln-Lys-Gly-Gln-Val-Thr-Arg-X₇ (SEQ ID NO: 17), wherein X₃ is selected from the group consisting of Phe, Tyr, His, Leu, Ala, and NorVal; and X₇ is absent or represents a positively charged amino acid residue, optionally conjugated with a label.

In some embodiments, the peptide or peptidomimetic comprises a sequence selected from the group consisting of:

(SEQ ID NO: 18)
Gly-Arg-Asn-Phe-Gln-Lys-Gly-Gln-Val-Thr-Arg;
(SEQ ID NO: 19)
Gly-Arg-Asn-His-Gln-Lys-Gly-Gln-Val-Thr-Arg;
(SEQ ID NO: 20)
Gly-Arg-Asn-Leu-Gln-Lys-Gly-Gln-Val-Thr-Arg;
(SEQ ID NO: 21)
Gly-Arg-Asn-NorVal-Gln-Lys-Gly-Gln-Val-Thr-Arg;
(SEQ ID NO: 22)
Gly-Arg-Asn-Phe-Gln-Lys-Gly-Gln-Val-Thr-Arg-
Lys(Z);
(SEQ ID NO: 23)
Gly-Arg-Asn-Ala-Gln-Lys-Gly-Gln-Val-Thr-Arg;
(SEQ ID NO: 24)
Gly-Arg-Asn-Tyr-Gln-Lys-Gly-Gln-Val-Thr-Arg;
and
(SEQ ID NO: 25)
Gly-Arg-Asn-Tyr-Gln-Lys-Gly-Gln-Val-Thr-Arg-
Lys(Z),

wherein Z is as defined above.

In some embodiments, the peptide consists of a sequence selected from the group consisting of:

(SEQ ID NO: 18)
Gly-Arg-Asn-Phe-Gln-Lys-Gly-Gln-Val-Thr-Arg;
(SEQ ID NO: 19)
Gly-Arg-Asn-His-Gln-Lys-Gly-Gln-Val-Thr-Arg;
(SEQ ID NO: 20)
Gly-Arg-Asn-Leu-Gln-Lys-Gly-Gln-Val-Thr-Arg;
(SEQ ID NO: 21)
Gly-Arg-Asn-NorVal-Gln-Lys-Gly-Gln-Val-Thr-Arg;
(SEQ ID NO: 22)
Gly-Arg-Asn-Phe-Gln-Lys-Gly-Gln-Val-Thr-Arg-
Lys(Z);
(SEQ ID NO: 23)
Gly-Arg-Asn-Ala-Gln-Lys-Gly-Gln-Val-Thr-Arg;
(SEQ ID NO: 24)
Gly-Arg-Asn-Tyr-Gln-Lys-Gly-Gln-Val-Thr-Arg;
and
(SEQ ID NO: 25)
Gly-Arg-Asn-Tyr-Gln-Lys-Gly-Gln-Val-Thr-Arg-
Lys(Z),

wherein Z is as defined above.

In some embodiments, the peptide or peptidomimetic comprises the sequence X₁-Phe-Thr-Lys-Gly-Gln-Val-Thr (SEQ ID NO: 26), wherein X₁ is any amino acid residue.

In some embodiments, the peptide or peptidomimetic comprises the sequence Gly-Phe-Thr-Lys-Gly-Gln-Val-Thr (SEQ ID NO: 27).

In additional embodiments, the peptide consists of the sequence Gly-Phe-Thr-Lys-Gly-Gln-Val-Thr (SEQ ID NO: 27).

In some embodiments, the peptide consists of 7-15 amino acids.

In some embodiments, the amino terminus of the peptide or peptidomimetic is modified with an amino-terminal blocking group selected from the group consisting of an acyl, alkyl and aryl. Each possibility represents a separate embodiment of the present invention.

In some embodiments, the carboxy terminus of the peptide or peptidomimetic is modified with a carboxy-terminal group selected from the group consisting of an amide, ester and alcohol group. Each possibility represents a separate embodiment of the present invention.

In some embodiments, the peptide or peptidomimetic is conjugated to at least one moiety selected from the group consisting of a permeability-enhancing moiety, a detectable label and a carrier.

In some embodiments, the conjugated peptide is according to Formula I as described above, wherein R₁ and R₂ are as defined above, and wherein:

X₁ is any amino acid residue;

X₂ is absent or represents a stretch of two amino acid residues selected from the group consisting of: Arg-X₈, wherein X₈ is any amino acid residue, Asp-Met and Leu-Gln;

X₃ is selected from the group consisting of Phe, Tyr, His, Leu, Ala, Ser and NorVal;

X₄ is selected from Thr and Gln;

X₅ is selected from Val and Ser;

X₆ is absent or selected from the group consisting of a positively charged amino acid residue, Ser and Val; and

X₇ is absent or represents a positively charged amino acid residue, optionally modified with a label. In some embodiments, the label is a detectable label.

In some embodiments, the conjugated peptide is according to Formula Ia:

R₁-X₁-Arg-Asn-X₃-Gln-Lys-Gly-Gln-Val-Thr-Arg-X₇-R₂,

wherein R₁, R₂, X₁, X₃ and X₇ are as defined above.

In some embodiments, the conjugated peptide is according to the following formula:

R₁-Gly-Arg-Asn-X₃-Gln-Lys-Gly-Gln-Val-Thr-Arg-X₇-R₂,

wherein R₁, R₂, X₃ and X₇ are as defined above.

In some embodiments, the peptide is according to any one of SEQ ID NOs: 28-39.

In some embodiments, R₁ is a permeability-enhancing moiety. In some embodiments, the permeability-enhancing moiety is a fatty acid residue.

In some embodiments, the fatty acid residue is a C12-C20 fatty acid.

In some embodiments, the fatty acid residue is a myristoyl (Myr).

In some embodiments, the fatty acid residue is a stearoyl (Stear).

In some embodiments, the fatty acid residue is a palmitoyl (Palm).

In some embodiments, R₂ is a carboxyl group selected from the group consisting of an amide, ester and alcohol group.

In some embodiments, R₂ is an amide group.

In some embodiments, the peptide consists of 7-15 amino acids.

In some embodiments, the step of administering is carried out via oral administration.

In some embodiments, the step of administering is carried out via parenteral administration.

The present invention further provides a method of suppression, prevention or treatment of complications of T2D, comprising administering to a patient in need of such treatment a pharmaceutical composition comprising at least one peptide, peptidomimetic or conjugate as defined above.

T2D complications which may be prevented, suppressed or treated according to the present invention, include but are not limited to: metabolic syndrome, fatty liver, insulin resistance, cancer, microvascular complications including neuropathy (nerve damage), nephropathy (kidney disease) and vision disorders (e.g., retinopathy, glaucoma, cataract and corneal disease), macrovascular complications including heart disease, stroke and peripheral vascular disease (which can lead to ulcers, gangrene and amputation).

Other complications of diabetes include infections, metabolic difficulties, impotence, autonomic neuropathy and pregnancy problems.

Treatment methods according to the present invention comprises, according to some specific embodiments, administration of at least one additional anti-diabetic agent.

According to some embodiments the at least one additional anti-diabetic agent is selected from the group consisting of: insulin, sufonylureas, alpha-glucosidase inhibitors, biguanides, meglitinides, and thiazolidinediones.

According to other embodiments the at least one additional anti-diabetic agent is selected from the group consisting of: sensitizers (such as biguanides and thiazolidinediones); secretagogues (such as sulfonylureas and nonsulfonylurea secretagogues); alpha-glucosidase inhibitors; peptide analogs (such as injectable incretin mimetics and injectable amylin analogues).

According to some particular embodiments, the anti-diabetic agent is selected from the group consisting of: metformin; rosiglitazone (Avandia™); pioglitazone (Actos™); tolbutamide (Orinase™); acetohexamide (Dymelor™); tolazamide (Tolinase™); chlorpropamide (Diabinese™); second-generation agents; glipizide (Glucotrol™); glyburide (Diabeta™, Micronase™, Glynas™e); glimepiride (Amaryl™); gliclazide (Diamicron™); rep aglinide (Prandin™); nateglinide (Starlix™); miglitol (Glyset™); acarbose (Precose™/Glucobay™); exenatide; liraglutide; vildagliptin (Galvus™); sitagliptin (Januvia™); saxagliptin (Onglyza™) linagliptin (Tradjenta™)

According other embodiments, the pharmaceutical composition of the present invention is administered to a subject in need thereof as part of a treatment regimen which does not include administration of other anti-diabetic agents.

The present invention provides, according to yet another aspect, a method for delaying the onset of T2D in subjects who are predisposed to the disease, comprising administering any pharmaceutical composition described above.

These and further aspects and features of the present invention will become apparent from the detailed description, examples and claims which follow.

### BRIEF DESCRIPTION OF THE FIGURES

FIG. 1. Inhibition of starvation-induced insulin-resistance by peptide ACD-047.7 (“047.7”, SEQ ID NO: 32, n=10), compared to two inactive peptides ACD-047.8 (“047.8”, SEQ ID NO: 67, n=9), and ACD-047.9 (“047.9”, SEQ ID NO: 68, n=10).

FIG. 2. Blood glucose in (db/db) mice following a 3-day treatment with peptide ACD-400.3 (SEQ ID NO: 29) compared to vehicle.

FIG. 3. Blood glucose in (db/db) mice with server diabetes following a 7-day treatment with peptide ACD-400.3 (SEQ ID NO: 29) compared to vehicle.

### DETAILED DESCRIPTION OF THE INVENTION

The present invention is directed according to some aspects to novel peptides and peptidomimetics derived from HO-1, HIF1α or DQX1. The present invention is further directed to pharmaceutical compositions comprising the peptides and use thereof in the treatment of Type 2 diabetes in subjects in need thereof.

A set of peptides was designed, derived from a segment of human HO-1 containing a natural lysine residue that was shown to be acetylated under certain circumstances, or from homolog sequences found in HIF1α and DQX1. Without wishing to be bound by any particular theory of a mechanism of action, it is contemplated that such peptides are capable of competitively inhibiting the post-translational acetylation of HO-1, thereby maintaining HO-1 in an active form. It is contemplated that maintaining HO-1 in an active form prevents accumulation of heme and subsequent downstream processes leading to development of insulin resistance and Type 2 diabetes. As exemplified herein below, such peptides were able to successfully inhibit insulin-resistance in normal fasting mice and lower blood glucose levels in obese diabetic mice.

As used herein, a “subject” is a mammal, typically a human. The subject may be a subject already diagnosed with Type 2 diabetes. For example, it may be a subject showing elevated blood glucose levels (e.g. fasting glucose and/or following a glucose tolerance test) if proper diet is not maintained and/or medications are not consumed. The subject may optionally also show overweight or obesity, hypertension, elevated triglycerides in the blood, elevated LDL-cholesterol in the blood and/or other symptoms associated with Type 2 diabetes. Alternatively, the subject may be a subject at risk of developing Type 2 diabetes. For example, it may be a subject that shows normal blood glucose level but is overweight or obese, and optionally also has at least one of the aforementioned symptoms such as hypertension.

As used herein, “treating” encompass reduction, amelioration or even elimination of at least some of the symptoms associated with the disease. For example, treatment may include lowering blood glucose levels to healthy normal range (fasting glucose and/or following a glucose tolerance test). Treatment may also include balancing the level of glucose in the blood and maintaining a balanced level of glucose in the blood. “Treatment” may also encompass prophylactic treatment. For example, treatment may include preventing development of hyperglycemia.

The term “level” as used herein refers to the amount of a certain substance contained in a sample (e.g., in blood sample). Typically, the term refers to the concentration of a certain substance in a sample (for example, amount in mg per unit volume of blood, such as mg per milliliter, or mg per deciliter).

As used herein, the terms “reducing”, “decreasing” and “lowering”, when referring to a level of a certain substance are intended to refer to reduction compared to an initial level, prior to treatment with the peptides as disclosed herein.

As used herein, the terms “balancing” and “balanced”, when referring to a level of a certain substance, are intended to describe a level that is within the normal range, that is considered healthy, as known in the art.

As used herein “peptide” indicates a sequence of amino acids linked by peptide bonds. Peptides according to some embodiments of the present invention consist of 6-20 amino acids, for example 7-20 amino acids or 7-15 amino acids.

In some embodiments, a peptide according to the present invention is up to 27 amino acids, for example up to 26 amino acids, 25 amino acids, 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, or up to 7 amino acids. Each possibility represents a separate embodiment of the invention.

The term “amino acid” refers to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine (Orn), canavanine, djenkolic acid, β-cyanoalanine), and synthetically derived α-amino acids, such as aminoisobutyric acid, norleucine (Nle), norvaline (NorVal, Nva), homocysteine and homoserine. β-Alanine and γ-amino butyric acid are examples of 1,3 and 1,4-amino acids, respectively, and many others as well known to the art.

Some of the amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by one-letter codes or three-letter codes according to IUPAC conventions. When there is no indication, the L isomer was used. The D isomers are indicated by “D” or “(D)” before the residue abbreviation.

As used herein, an “amino acid residue” means the moiety which remains after the amino acid has been conjugated to additional amino acid(s) to form a peptide, or to a moiety (such as a permeability-enhancing moiety), typically through the alpha-amino and carboxyl of the amino acid.

As used herein, a “fatty acid residue” means the moiety which remains after the fatty acid has been conjugated to the amino acid (directly or through a linker).

As used herein, the term “label” refers to a moiety attached to an amino acid residue within a peptide, peptidomimetic or conjugate according to the present invention, typically at the terminus (N- or C-) of the peptide, peptidomimetic or conjugate, which: (i) facilitates detection of the peptide, peptidomimetic or conjugate (namely, a detectable label), for example, a dye, a fluorescent agent, an enzyme, a specific binding pair component such as avidin/biotin and the like; (ii) facilitates capture of the peptide, peptidomimetic or conjugate e.g. to a solid substrate, such as biotin, haptens and the like; and/or (iii) affects solubility or modifies cellular uptake, e.g., cell permeability enhancing moieties such as fatty acid residues and the like. Each possibility represents a separate embodiment of the present invention.

In some embodiments, the label is a detectable label. In some embodiments, the label is a permeability-enhancing moiety.

Peptides

In some embodiments, a synthetic peptide or peptidomimetic provided herein comprises the sequence X₁-X₂-X₃-X₄-Lys-Gly-Gln-X₅-Thr-X₆-X₇ (SEQ ID NO: 1), wherein:

X₁ is an amino acid residue other than Met and His;

X₂ is absent or represents a stretch of two amino acid residues selected from the group consisting of: Arg-X₈, wherein X₈ is any amino acid residue, Asp-Met and Leu-Gln;

X₃ is selected from the group consisting of Phe, Tyr, His, Leu, Ala, Ser and NorVal;

X₄ is selected from Thr and Gln;

X₅ is selected from Val, Thr and Ser;

X₆ is absent or selected from the group consisting of a positively charged amino acid residue, Ser and Val; and

X₇ is absent or represents a positively charged amino acid residue, optionally modified with a moiety, e.g. a detectable label.

In some embodiments, X₂ represents a stretch of two amino acid residues selected from the group consisting of Arg-Asn, Asp-Met, Leu-Gln and Arg-Gly.

In some embodiments, the peptide or peptidomimetic comprises or consists of a sequence selected from the group consisting of:

(SEQ ID NO: 2)
X₁-Arg-Asn-Phe-Gln-Lys-Gly-Gln-Val-Thr-Arg;
(SEQ ID NO: 3)
X₁-Asp-Met-Phe-Thr-Lys-Gly-Gln-Val-Thr-Arg;
(SEQ ID NO: 4)
X₁-Leu-Gln-Ser-Thr-Lys-Gly-Gln-Ser-Thr-Ser;
(SEQ ID NO: 5)
X₁-Leu-Gln-Ser-Gln-Lys-Gly-Gln-Ser-Thr-Ser;
and
(SEQ ID NO: 6)
X₁-Arg-Gly-Phe-Gln-Lys-Gly-Gln-Val-Thr-Val,

wherein X₁ is an amino acid residue other than Met and His. Each possibility represents a separate embodiment of the present invention.

In some embodiments, a synthetic peptide or peptidomimetic provided herein comprises the sequence X₁-X₂-X₃-X₄-Lys-Gly-Gln-Val-Thr-X₆-X₇ (SEQ ID NO: 7), wherein:

X₁ is absent or is an amino acid residue other than Met;

X₂ is absent or represents Arg-Asn;

X₃ is selected from the group consisting of Phe, Tyr, His, Leu, Ala and NorVal;

X₄ is Thr or Gln;

X₆ is absent or represents Arg; and

X₇ is absent or represents any amino acid residue, optionally modified with a label, such as a detectable label.

In some embodiments, X₁ is Gly. According to these embodiments, the peptide or peptidomimetic comprises the sequence Gly-X₂-X₃-X₄-Lys-Gly-Gln-Val-Thr-X₆-X₇ (SEQ ID NO: 8), wherein X₂, X₃, X₄, X₅ and X₆ are as defined above.

In some embodiments, X₇ is absent. In other embodiments, X₇ is an amino acid residue modified with a label. In other embodiments, X₇ is a positively charged amino acid residue modified with a label. In some embodiments, X₇ is Lys(Z), wherein Z is the label connected to the epsilon amino group of the Lys residue. In some embodiments, the label is a detectable label. In some particular embodiments, Z is biotin. In additional particular embodiments, Z is a dansyl moiety.

In some embodiments, X₁-X₂-X₃-X₄ represent a stretch of amino acid residues selected from the group consisting of:

(SEQ ID NO: 9)
Gly-Arg-Asn-Phe-Gln;
(SEQ ID NO: 10)
Gly-Arg-Asn-His-Gln;
(SEQ ID NO: 11)
Gly-Arg-Asn-Leu-Gln;
(SEQ ID NO: 12)
Gly-Arg-Asn-NorVal-Gln;
(SEQ ID NO: 13)
Gly-Arg-Asn-Ala-Gln;
(SEQ ID NO: 14)
Gly-Arg-Asn-Tyr-Gln;
and
(SEQ ID NO: 15)
Gly-Phe-Thr.

In some embodiments, X₁-X₂-X₃-X₄ represent Gly-Arg-Asn-Phe-Gln (SEQ ID NO: 9). In other embodiments, X₁-X₂-X₃-X₄ represent Gly-Arg-Asn-His-Gln (SEQ ID NO: 10). In additional embodiments, X₁-X₂-X₃-X₄ represent Gly-Arg-Asn-Leu-Gln (SEQ ID NO: 11). In yet additional embodiments, X₁-X₂-X₃-X₄ represent Gly-Arg-Asn-NorVal-Gln (SEQ ID NO: 12). In yet additional embodiments, X₁-X₂-X₃-X₄ represent Gly-Arg-Asn-Ala-Gln (SEQ ID NO: 13). In yet additional embodiments, X₁-X₂-X₃-X₄ represent Gly-Arg-Asn-Tyr-Gln (SEQ ID NO: 14).

In some embodiments, X₁-X₂-X₃-X₄ represent Gly-Phe-Thr (SEQ ID NO: 15).

In some embodiments, X₅-X₆ represent Arg or Arg-Lys(Z), wherein Z is a detectable label.

In other embodiments, X₅-X₆ are absent.

In some embodiments, the peptide or peptidomimetic comprises the sequence X₁-Arg-Asn-X₃-Gln-Lys-Gly-Gln-Val-Thr-Arg-X₇ (SEQ ID NO: 16), wherein X₁, X₃ and X₇ are as defined above.

In some embodiments, the peptide or peptidomimetic comprises the sequence Gly-Arg-Asn-X₃-Gln-Lys-Gly-Gln-Val-Thr-Arg-X₇ (SEQ ID NO: 17), wherein X₃ and X₇ are as defined above.

In some embodiments, the peptide or peptidomimetic comprises the sequence Gly-Arg-Asn-Phe-Gln-Lys-Gly-Gln-Val-Thr-Arg (SEQ ID NO: 18).

In some embodiments, the peptide or peptidomimetic comprises the sequence Gly-Arg-Asn-His-Gln-Lys-Gly-Gln-Val-Thr-Arg (SEQ ID NO: 19).

In some embodiments, the peptide or peptidomimetic comprises the sequence Gly-Arg-Asn-Leu-Gln-Lys-Gly-Gln-Val-Thr-Arg (SEQ ID NO: 20).

In some embodiments, the peptide or peptidomimetic comprises the sequence Gly-Arg-Asn-NorVal-Gln-Lys-Gly-Gln-Val-Thr-Arg (SEQ ID NO: 21).

In some embodiments, the peptide or peptidomimetic comprises the sequence Gly-Arg-Asn-Phe-Gln-Lys-Gly-Gln-Val-Thr-Arg-Lys(Z) (SEQ ID NO: 22).

In some embodiments, the peptide or peptidomimetic comprises the sequence Gly-Arg-Asn-Ala-Gln-Lys-Gly-Gln-Val-Thr-Arg (SEQ ID NO: 23).

In some embodiments, the peptide or peptidomimetic comprises the sequence Gly-Arg-Asn-Tyr-Gln-Lys-Gly-Gln-Val-Thr-Arg (SEQ ID NO: 24).

In some embodiments, the peptide or peptidomimetic comprises the sequence Gly-Arg-Asn-Tyr-Gln-Lys-Gly-Gln-Val-Thr-Arg-Lys(Z) (SEQ ID NO: 25), wherein Z is as defined above.

In some embodiments, the peptide or peptidomimetic consists of a sequence selected from the group consisting of SEQ ID NOs: 18-25. Each possibility represents a separate embodiment of the present invention.

In some embodiments, the peptide or peptidomimetic comprises the sequence X₁-Phe-Thr-Lys-Gly-Gln-Val-Thr (SEQ ID NO: 26), wherein X₁ is as defined above.

In some embodiments, the peptide or peptidomimetic comprises the sequence Gly-Phe-Thr-Lys-Gly-Gln-Val-Thr (SEQ ID NO: 27). In some embodiments, the peptide or peptidomimetic consists of the sequence Gly-Phe-Thr-Lys-Gly-Gln-Val-Thr (SEQ ID NO: 27).

In some embodiments, a peptide or peptidomimetic provided herein comprises or consists of the sequence X₁-Arg-Asn-X₃-X₄-Lys-Gly-Gln-Val-Thr-Arg (SEQ ID NO: 41), wherein X₁ is any amino acid and X₃, X₄ are as defined above.

In some embodiments, a peptide or peptidomimetic provided herein comprises or consist of the sequence Arg-Asn-X₃-X₄-Lys-Gly-Gln-Val-Thr-Arg (SEQ ID NO: 42), wherein X₃, X₄ are as defined above.

In some embodiments, the present invention provides a synthetic or recombinant peptide or peptidomimetic of 7-27 amino acids, the peptide comprising the sequence X₁-X₂-X₃-X₄-X₅-Gly-Gln-Val-X₆ (SEQ ID NO: 43), wherein:

X₁ and X₆ are each independently a stretch of 0-10 amino acid residues;

X₂ is an amino acid residue other than Met;

X₃ is selected from the group consisting of Phe, Tyr, His, Leu and NorVal;

X₄ is Thr or Gln; and

X₅ is selected from the group consisting of Lys, D-Lys, Ac(ε)-Lys, Arg and Orn.

In some embodiments, X₁ and X₆ are each independently a stretch of 0-5 amino acid residues. In additional embodiments, X₁ and X₆ are each independently a stretch of 1-3 amino acid residues.

In some embodiments, X₁ comprises Gly. In additional embodiments, X₁ comprises Arg.

In some embodiments, X₁ is selected from the group consisting of Gly, Gly-Arg, and Gly-Gln-Phe-Nle-Arg (SEQ ID NO: 44). Each possibility represents a separate embodiment of the present invention.

In some embodiments, X₂ is selected from the group consisting of Asn, Gly and Nle. Each possibility represents a separate embodiment of the present invention.

In some embodiments, X₃ is Phe or Tyr. In additional embodiments, X₃ is selected from the group consisting of His, Leu and NorVal. Each possibility represents a separate embodiment of the present invention.

In some embodiments, X₃-X₄-X₅ represent Phe-Thr-Lys. In other embodiments, X₃-X₄-X₅ represent Phe-Gln-Lys. In additional embodiments, X₃-X₄-X₅ represent His-Gln-Lys. In yet additional embodiments, X₃-X₄-X₅ represent Leu-Gln-Lys. In yet additional embodiments, X₃-X₄-X₅ represent NorVal-Gln-Lys.

In some embodiments, X₆ comprises Thr.

In some embodiments, X₆ is selected from the group consisting of Thr, Thr-Thr, Thr-Arg, Thr-X₇ and Thr-Arg-X₇, wherein X₇ is an amino acid residue modified with a detectable label. Each possibility represents a separate embodiment of the present invention.

In some embodiments, the peptide consists of 7-20 amino acids. In additional embodiments, the peptide consists of 7-15 amino acids.

In some embodiments, there is provided herein a synthetic or recombinant peptide or peptidomimetic of 7-27 amino acids, the peptide comprising the sequence X₁-X₂-X₃-X₄-X₅-Gly-Gln-Val-X₆ (SEQ ID NO: 43), wherein:

X₁ and X₆ are each independently a stretch of 0-10 amino acid residues;

X₂ is an amino acid residue other then Met;

X₃ is selected from: (i) an aromatic amino acid residue, for example Phe or Tyr; (ii) a non-polar amino acid residue, for example Leu and NorVal; and (iii) His;

X₄ is a polar/hydrophilic amino acid residue, for example, Thr or Gln; and

X₅ is a basic amino acid residue for example selected from the group consisting of Lys, D-Lys, Arg and Orn.

In some embodiments, X₃ is an aromatic amino acid residue, for example Phe or Tyr. In other embodiments, X₃ is a non-polar amino acid residue, for example Leu and NorVal. In additional embodiments, X₃ is His.

Conjugates

In some embodiments, there is provided herein a conjugate comprising the peptide or peptidomimetic of the invention and at least one moiety selected from the group consisting of a permeability-enhancing moiety, a detectable label and a carrier.

In some embodiments, the conjugate comprises the peptide or peptidomimetic of the invention and a permeability-enhancing moiety. In other embodiments, the conjugate comprises the peptide or peptidomimetic of the invention and detectable label. In additional embodiments, the conjugate comprises the peptide or peptidomimetic of the invention and a carrier.

In some embodiments, there is provided herein a conjugate according to Formula I:

R₁-X₁-X₂-X₃-X₄-Lys-Gly-Gln-X₅-Thr-X₆-X₇-R₂,

wherein R₁ is selected from the group consisting of a permeability-enhancing moiety and a detectable moiety, linked via a direct bond or via a linker; R₂ designates OH of an unmodified carboxy terminal group or a modified carboxy terminal group; and X₁-X₇ are as defined above.

In some embodiments, R₁ is a permeability-enhancing moiety linked via a direct bond. In other embodiments, R₁ is a permeability-enhancing moiety linked via a linker.

In some embodiments, the permeability-enhancing moiety is a fatty acid residue.

In some embodiments, R₂ is a modified carboxy terminal group selected from the group consisting of an amide, ester and alcohol group. Each possibility represents a separate embodiment of the present invention.

In some embodiments, the peptide in the conjugate consists of 7-15 amino acids.

In some embodiments, the conjugate is according to Formula Ia:

R₁-X₁-Arg-Asn-X₃-Gln-Lys-Gly-Gln-Val-Thr-Arg-X₇-R₂,

wherein R₁, R₂, X₁, X₃ and X₇ are as defined above.

In some embodiments, the conjugate is according to the following formula:

R₁-Gly-Arg-Asn-X₃-Gln-Lys-Gly-Gln-Val-Thr-Arg-X₇-R₂,
wherein R₁, R₂, X₁, X₃ and X₇ are as defined above.

In some embodiments, the conjugate is R₁-Gly-Arg-Asn-Phe-Gln-Lys-Gly-Gln-Val-Thr-Arg-R₂, wherein R₁ and, R₂, are as defined above.

In some embodiments, the conjugate is R₁-Gly-Arg-Asn-His-Gln-Lys-Gly-Gln-Val-Thr-Arg-R₂, wherein R₁ and, R₂, are as defined above.

In some embodiments, the conjugate is R₁-Gly-Arg-Asn-Leu-Gln-Lys-Gly-Gln-Val-Thr-Arg-R₂, wherein R₁ and, R₂, are as defined above.

In some embodiments, the conjugate is R₁-Gly-Arg-Asn-NorVal-Gln-Lys-Gly-Gln-Val-Thr-Arg-R₂, wherein R₁ and, R₂, are as defined above.

In some embodiments, the conjugate is R₁-Gly-Arg-Asn-Phe-Gln-Lys-Gly-Gln-Val-Thr-Arg-Lys(Z)-R₂, wherein R₁, R₂, and Z are as defined above.

In some embodiments, the conjugate is R₁-Gly-Arg-Asn-Ala-Gln-Lys-Gly-Gln-Val-Thr-Arg-R₂, wherein R₁ and, R₂, are as defined above.

In some embodiments, the conjugate is R₁-Gly-Arg-Asn-Tyr-Gln-Lys-Gly-Gln-Val-Thr-Arg-R₂, wherein R₁ and, R₂, are as defined above.

In some embodiments, the conjugate is R₁-Gly-Arg-Asn-Tyr-Gln-Lys-Gly-Gln-Val-Thr-Arg-Lys(Z)-R₂, wherein R₁, R₂, and Z are as defined above.

In some particular embodiments, the conjugate is Myr-Gly-Arg-Asn-Phe-Gln-Lys-Gly-Gln-Val-Thr-Arg-NH₂ (SEQ ID NO: 28).

In additional particular embodiments, the conjugate is Stear-Gly-Arg-Asn-Phe-Gln-Lys-Gly-Gln-Val-Thr-Arg-NH₂, (SEQ ID NO: 29).

In additional particular embodiments, the conjugate is Palm-Gly-Arg-Asn-His-Gln-Lys-Gly-Gln-Val-Thr-Arg-NH₂ (SEQ ID NO: 30).

In additional particular embodiments, the conjugate is Stear-Gly-Arg-Asn-His-Gln-Lys-Gly-Gln-Val-Thr-Arg-NH₂ (SEQ ID NO: 31).

In additional particular embodiments, the conjugate is Myr-Gly-Arg-Asn-Leu-Gln-Lys-Gly-Gln-Val-Thr-Arg-NH₂ (SEQ ID NO: 32).

In additional particular embodiments, the conjugate is Myr-Gly-Arg-Asn-NorVal-Gln-Lys-Gly-Gln-Val-Thr-Arg-NH₂ (SEQ ID NO: 33).

In additional particular embodiments, the conjugate is Myr-Gly-Arg-Asn-Phe-Gln-Lys-Gly-Gln-Val-Thr-Arg-Lys(Biotin)-NH₂ (SEQ ID NO: 34).

In additional particular embodiments, the conjugate is Myr-Gly-Arg-Asn-Ala-Gln-Lys-Gly-Gln-Val-Thr-Arg-NH₂ (SEQ ID NO: 35).

In additional particular embodiments, the conjugate is Palm-Gly-Arg-Asn-Phe-Gln-Lys-Gly-Gln-Val-Thr-Arg-NH₂ (SEQ ID NO: 36).

In additional particular embodiments, the conjugate is Stear-Gly-Arg-Asn-Tyr-Gln-Lys-Gly-Gln-Val-Thr-Arg-NH₂ (SEQ ID NO: 37).

In additional particular embodiments, the conjugate is Stear-Gly-Arg-Asn-Tyr-Gln-Lys-Gly-Gln-Val-Thr-Arg-Lys(Dansyl)-NH₂ (SEQ ID NO: 38).

In some embodiments, the conjugate is according to Formula Ib:

R₁-X₁-Phe-Thr-Lys-Gly-Gln-Val-Thr-R₂,

wherein R₁, R₂ and X₁ are as defined above.

In some embodiments, the conjugate is R₁-Gly-Phe-Thr-Lys-Gly-Gln-Val-Thr-R₂, wherein R₁ and R₂ are as defined above.

In some particular embodiments, the conjugate is Myr-Gly-Phe-Thr-Lys-Gly-Gln-Val-Thr-NH₂ (SEQ ID NO: 39).

In some embodiments, there is provided herein a conjugate according to Formula II:

R₁-X₁-X₂-X₃-X₄-X₅-Gly-Gln-Val-X₆-R₂,

wherein R₁ is selected from the group consisting of a permeability-enhancing moiety and a detectable moiety, linked via a direct bond or via a linker; R₂ designates OH of an unmodified carboxy terminal group or a modified carboxy terminal group; and X₁-X₆ are as defined above.

In some embodiments, the conjugate is selected from the group consisting of:

R₁-X₁ₐ-Arg-Asn-Phe-Gln-Lys-Gly-Gln-Val-Thr-Arg-X₆ₐ-R₂; and (i)

R₁-X_{1b}-X₂-Phe-Thr-Lys-Gly-Gln-Val-Thr-X_{6b}-R₂, (ii)

wherein:
X₁ₐ is a stretch of 0-9 amino acid residues;
X₆ₐ is a stretch of 0-8 amino acid residues;
X_{1b} is a stretch of 0-10 amino acid residues;
X_{6b} is a stretch of 0-9 amino acid residues; and
   R₁, R₂ and X₂ are as defined above.

In some embodiments, the conjugate is selected from the group consisting of:

R₁-Gly-Arg-Asn-Phe-Gln-Lys-Gly-Gln-Val-Thr-Arg-R₂,

R₁-Gly-Arg-Asn-Phe-Gln-(D-Lys)-Gly-Gln-Val-Thr-Arg-R₂;

R₁-Gly-Arg-Asn-Phe-Gln-Arg-Gly-Gln-Val-Thr-Arg-R₂;

R₁-Gly-Arg-Asn-Phe-Gln-Orn-Gly-Gln-Val-Thr-Arg-R₂;

R₁-Gly-Arg-Asn-Phe-Gln-(Ac-Lys)-Gly-Gln-Val-Thr-Arg-R₂;

R₁-Gly-Arg-Asn-Tyr-Gln-Lys-Gly-Gln-Val-Thr-Arg-R₂;

R₁-Gly-Phe-Gln-Lys-Gly-Gln-Val-Thr-Arg-R₂;

R₁-Gly-Arg-Asn-Phe-Gln-Lys-Gly-Gln-Val-R₂;

R₁-Gly-Gln-Phe-Nle-Arg-Asn-Phe-Gln-Lys-Gly-Gln-Val-Thr-Arg-R₂; and

R₁-Gly-Arg-Asn-Phe-Gln-Lys-Gly-Gln-Val-Thr-Arg-Lys(Z)-R₂,

wherein Z is a detectable label. Each possibility represents a separate embodiment of the present invention.

In some embodiments, the conjugate is R₁-Gly-Arg-Asn-Phe-Gln-Lys-Gly-Gln-Val-Thr-Arg-R₂.

In some embodiments, the conjugate is selected from the group consisting of:

(SEQ ID NO: 28)
Myr-Gly-Arg-Asn-Phe-Gln-Lys-Gly-Gln-Val-Thr-Arg-
NH₂;
(SEQ ID NO: 45)
Myr-Gly-Arg-Asn-Tyr-Gln-Lys-Gly-Gln-Val-Thr-Arg-
NH₂;
(SEQ ID NO: 46)
Myr-Gly-Phe-Gln-Lys-Gly-Gln-Val-Thr-Arg-NH2;
(SEQ ID NO: 47)
RhodaminB-Gly-Arg-Asn-Phe-Gln-Lys-Gly-Gln-Val-
Thr-Arg-NH₂,
(SEQ ID NO: 48)
Myr-Gly-Gln-Phe-Nle-Arg-Asn-Phe-Gln-Lys-Gly-Gln-
Val-Thr-Arg-NH₂;
and
(SEQ ID NO: 34)
Myr-Gly-Arg-Asn-Phe-Gln-Lys-Gly-Gln-Val-Thr-Arg-
Lys(Biotin)-NH₂.

Each possibility represents a separate embodiment of the present invention.

In some embodiments, the conjugate is selected from the group consisting of:

R₁-Gly-Phe-Thr-Lys-Gly-Gln-Val-Thr-R₂;

R₁-Gly-Nle-Phe-Thr-Lys-Gly-Gln-Val-Thr-R₂;

R₁-Gly-Phe-Thr-Lys-Gly-Gln-Val-Thr-Thr-R₂;

R₁-Gly-Phe-Thr-Lys-Gly-Gln-Val-Thr-Lys(Z)-R₂,

wherein Z is a detectable label; and

R₁-Gly-Arg-Asn-Phe-Thr-Lys-Gly-Gln-Val-Thr-Arg-R₂.

Each possibility represents a separate embodiment of the present invention.

In some embodiments, the conjugate is R₁-Gly-Phe-Thr-Lys-Gly-Gln-Val-Thr-R₂.

In some embodiments, the conjugate is selected from the group consisting of:

(SEQ ID NO: 39)
Myr-Gly-Phe-Thr-Lys-Gly-Gln-Val-Thr-NH₂;
(SEQ ID NO: 49)
Myr-Gly-Nle-Phe-Thr-Lys-Gly-Gln-Val-Thr-NH₂;
(SEQ ID NO: 50)
Myr-Gly-Phe-Thr-Lys-Gly-Gln-Val-Thr-Thr-NH₂;
(SEQ ID NO: 51)
Myr-Gly-Phe-Thr-Lys-Gly-Gln-Val-Thr-Lys(Biotin)-NH₂;
(SEQ ID NO: 52)
RhodamineB-Gly-Phe-Thr-Lys-Gly-Gln-Val-Thr-NH₂;
and
(SEQ ID NO: 53)
Myr-Gly-Arg-Asn-Phe-Thr-Lys-Gly-Gln-Val-Thr-Arg-NH₂.

Each possibility represents a separate embodiment of the present invention.

In some embodiments, the conjugate is R₁-Gly-Arg-Asn-X₃ₐ-Gln-Lys-Gly-Gln-Val-Thr-Arg-R₂, wherein X₃ₐ is selected from the group consisting of His, Leu and NorVal, and R₁ and R₂ are as defined above.

In some embodiments, the conjugate is selected from the group consisting of:

(SEQ ID NO: 54)
Stear-Gly-Arg-Asn-His-Gln-Lys-Gly-Gln-Val-Thr-Arg-
NH₂;
(SEQ ID NO: 32)
Myr-Gly-Arg-Asn-Leu-Gln-Lys-Gly-Gln-Val-Thr-Arg-
NH₂;
and
(SEQ ID NO: 33)
Myr-Gly-Arg-Asn-NorVal-Gln-Lys-Gly-Gln-Val-Thr-Arg-
NH₂.

Each possibility represents a separate embodiment of the present invention.

In some embodiments, the conjugate is Stear-Gly-Arg-Asn-His-Gln-Lys-Gly-Gln-Val-Thr-Arg-NH₂ (SEQ ID NO: 54).

In some embodiments, there is provided herein an isolated peptide or peptidomimetic of 6-26 amino acids selected from the group consisting of:

(i) a peptide according to Formula III: R₁-Xₐ-X_{b}-Gln-X_{c}-Gly-Gln-Val-X_{d}-R₂, wherein:

R₁ designates a hydrogen of an unmodified amino terminal group or is selected from the group consisting of an amino terminal blocking group, a permeability-enhancing moiety, a detectable label and a carrier;

Xₐ and X_{d} each independently is a stretch of 0-10 amino acid residues;

X_{b} is selected from: (i) an aromatic amino acid residue, for example Phe or Tyr;

(ii) a non-polar amino acid residue, for example Leu and NorVal; and (iii) His;

X_{c} is a basic amino acid, for example selected from the group consisting of Lys, D-Lys, Arg and Ornithine (Orn); and

R₂ designates OH of an unmodified carboxy terminal group or a modified carboxy terminal group,

and

(ii) a peptide according to Formula IV: R₁-Xₑ-X_{f}-Phe-Thr-X_{g}-Gly-Gln-Val-Thr-Xₕ-R₂, wherein:

R₁ designates a hydrogen of an unmodified amino terminal group or is selected from the group consisting of an amino terminal blocking group, a permeability-enhancing moiety, a detectable label and a carrier;

Xₑ and Xₕ each independently is a stretch of 0-10 amino acid residues;

X_{f} is an amino acid residue other than Met;

X_{g} is a basic amino acid, for example selected from the group consisting of Lys, D-Lys, Arg and Orn; and

R₂ designates OH of an unmodified carboxy terminal group or a modified carboxy terminal group.

In some embodiments, X_{b} is an aromatic amino acid residue, for example Phe or Tyr. In other embodiments, X_{b} is a non-polar amino acid residue, for example Leu and NorVal. In additional embodiments, X_{b} is His.

In some embodiments, Xₐ, X_{d}, Xₑ and Xₕ are each independently absent or a stretch of 1-5 amino acid residues. In some embodiments, Xₐ, X_{d}, Xₑ and Xₕ are each independently a stretch of 1-3 amino acid residues.

In some embodiments, Xₐ comprises Gly. In some embodiments, Xₐ is Gly. In some embodiments, Xₐ comprises Arg-Asn. In some embodiments, Xₐ is Arg-Asn. In some embodiments, Xₐ is Gly-Arg-Asn. In some embodiments, Xₐ is Gly-Gln-Phe-Nle (SEQ ID NO: 55).

In some embodiments, X_{d} comprises Thr-Arg. In some embodiments, X_{d} is Thr-Arg. In some embodiments, X_{d} is Thr-Arg-Lys(Biotin).

In some embodiments, Xₑ is absent. In other embodiments, Xₑ is Gly.

In some embodiments, X_{f} is Gly. In other embodiments, X_{f} is Nle.

In some embodiments, Xₕ is absent. In other embodiments, Xₕ is Thr. In yet other embodiments, Xₕ is Thr Lys(Biotin).

In some embodiments, the peptide is selected from the group consisting of:

R₁-Xₐ-Phe-Gln-Lys-Gly-Gln-Val-X_{d}-R₂, and (i)

R₁-Xₑ-X_{f}-Phe-Thr-Lys-Gly-Gln-Val-Thr-Xₕ-R₂, (ii)

wherein R₁, R₂, Xₐ, X_{d}, Xₑ, X_{f} and Xₕ are as defined above. Each possibility represents a separate embodiment of the present invention.

In some embodiments, the peptide is selected from the group consisting of:

R₁-Xₐ-Arg-Asn-Phe-Gln-Lys-Gly-Gln-Val-Thr-Arg-X_{d}-R₂, and (i)

R₁-Xₑ-X_{f}-Phe-Thr-Lys-Gly-Gln-Val-Thr-Xₕ-R₂, (ii)

wherein:

X_{a′} and X_{d′} are each a stretch of 0-8 amino acid residues; and

R₁, R₂, Xₑ, X_{f} and Xₕ are as defined above. Each possibility represents a separate embodiment of the present invention.

Additional peptides comprising the core sequences Phe-Thr-Lys-Gly-Gln-Val-Thr (SEQ ID NO: 56) or Arg-Asn-Phe-Gln-Lys-Gly-Gln-Val-Thr-Arg (SEQ ID NO: 57) are within the scope of the present invention as long as they differ from known sequences.

In some embodiments, the conjugated peptides of the present invention comprise a permeability-enhancing moiety.

In some embodiments, the amino terminal of the peptides disclosed herein is modified. In some embodiments, the amino terminal modification is addition of a permeability-enhancing moiety.

“Permeability” refers to the ability of an agent or substance to penetrate, pervade, or diffuse through a barrier or membrane, typically a phospholipid membrane. A “cell permeability”, “cell penetration” or “permeability-enhancing” moiety refers to a molecule which is able to facilitate or enhance penetration of molecules through membranes. Non-limitative examples of permeability-enhancing moieties include hydrophobic moieties such as lipids, fatty acids, steroids and bulky aromatic or aliphatic compounds.

In some embodiments, the permeability-enhancing moiety is covalently linked to the N-terminus of the peptide via a direct bond. In other embodiments, the permeability-enhancing moiety is covalently linked to the N-terminus of the peptide via a linker. In some embodiments, the permeability-enhancing moiety is a fatty acid residue. In some embodiments, the fatty acid residue is selected from C12-C20 fatty acids. In some particular embodiments, the fatty acid residue is a myristoyl group (Myr). In additional particular embodiments, the fatty acid residue is a stearoyl group (Steal). In yet additional embodiments, the fatty acid residue is a palmitoyl group (Palm).

In some embodiments, the amino terminal modification is addition of a detectable moiety or label. In some particular embodiments, the detectable moiety or label is Rhodamine B.

In some embodiments, the amino terminus is modified with an amino terminal blocking group. In some embodiments, the amino terminal blocking group is selected from the group consisting of an acetyl and alkyl. Each possibility represents a separate embodiment of the present invention.

In some embodiments, the carboxy terminus of the peptides disclosed herein is modified. In some embodiments, the carboxy terminus is modified with a carboxy terminal group. In some embodiments, the carboxy terminal group is selected from the group consisting of amide, ester and alcohol group. Each possibility represents a separate embodiment of the present invention. In some particular embodiments, the carboxy terminal group is an amide group.

The procedures utilized to construct peptide compounds of the present invention generally rely on the known principles and methods of peptide synthesis, such as solid phase peptide synthesis, partial solid phase synthesis, fragment condensation and classical solution synthesis.

Some of the peptides of the present invention, that do not comprise non-coded amino acids, can be synthesized using recombinant methods know in the art. Peptide conjugates may be synthesized chemically or alternatively may be produced recombinantly and coupled synthetically with the conjugating moiety.

The peptides of the invention can be used in the form of pharmaceutically acceptable salts. As used herein the term “salts” refers to both salts of carboxyl groups and to acid addition salts of amino or guanido groups of the peptide molecule. The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. Pharmaceutically acceptable salts include those salts formed with free amino groups such as salts derived from non-toxic inorganic or organic acids such as acetic acid, citric acid or oxalic acid and the like, and those salts formed with free carboxyl groups such as salts derived from non-toxic inorganic or organic bases such as sodium, calcium, potassium, ammonium, calcium, ferric or zinc, isopropylamine, triethylamine, procaine, and the like.

Analogs and derivatives of the peptides are also within the scope of the present application.

“Derivatives” of the peptides of the invention as used herein cover derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it, and do not adversely affect the immunogenic properties thereof.

These derivatives may include, for example, aliphatic esters of the carboxyl groups, amides of the carboxyl groups produced by reaction with ammonia or with primary or secondary amines, N-acyl derivatives of free amino groups of the amino acid residues, e.g., N-acetyl, formed by reaction with acyl moieties (e g, alkanoyl or carbocyclic aroyl groups), or O-acyl derivatives of free hydroxyl group (e.g., that of seryl or threonyl residues) formed by reaction with acyl moieties.

“Analogs” of the peptides of the invention as used herein cover compounds which have the amino acid sequence according to the invention except for one or more amino acid changes, typically, conservative amino acid substitutions.

In some embodiments, an analog has at least about 75% identity to the sequence of the peptide of the invention, for example at least about 80%, at least about 85%, at least about 90%, at least about 99% identity to the sequence of the peptide of the invention.

Conservative substitutions of amino acids as known to those skilled in the art are within the scope of the present invention. Conservative amino acid substitutions include replacement of one amino acid with another having the same type of functional group or side chain e.g. aliphatic, aromatic, positively charged, negatively charged.

Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K), Histidine (H);

5) Isoleucine (I), Leucine (L), Methionine (M), Valine (V); and

6) Phenylalanine (F), Tyrosine (Y), Tryptophan (W).

Analogs according to the present invention may comprise also peptidomimetics. “Peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with another covalent bond. A peptidomimetic according to the present invention may optionally comprise at least one bond which is an amide replacement bond such as urea bond, carbamate bond, sulfonamide bond, hydrazine bond, or any other covalent bond. The design of appropriate analogs may be computer assisted. Analogs are included in the invention as long as they remain pharmaceutically acceptable and their activity is not damaged

Pharmaceutical Compositions and Uses

The present invention further provides pharmaceutical compositions comprising a peptide, peptidomimetic or conjugate as disclosed herein and a pharmaceutically acceptable carrier, and optionally other pharmaceutically acceptable excipients.

In some embodiments, the pharmaceutical compositions are used for the treatment of T2D.

Treatment according to the present invention encompass administration of the pharmaceutical compositions of the present invention alone or in combination with any additional agent, composition or therapy use for prevention, alleviation or treatment of T2D, insulin resistance or metabolic syndrome, or of any complication thereof.

T2D complications which may be prevented, suppressed or treated according to the present invention, include but are not limited to: metabolic syndrome, fatty liver, insulin resistance, cancer, microvascular complications including neuropathy (nerve damage), nephropathy (kidney disease) and vision disorders (e.g., retinopathy, glaucoma, cataract and corneal disease), macrovascular complications including heart disease, stroke and peripheral vascular disease (which can lead to ulcers, gangrene and amputation). Each possibility represents a separate embodiment of the present invention.

Other complications of diabetes include infections, metabolic difficulties, impotence, autonomic neuropathy and pregnancy problems. Each possibility represents a separate embodiment of the present invention.

The pharmaceutical compositions are typically formulated for systemic administration. Suitable routes of administration include but are not limited to oral, rectal, buccal, nasal, intravenous, intraarticular, intramuscular, subcutaneous and intradermal. Each possibility represents a separate embodiment of the present invention.

The present invention further provides methods for treating Type 2 diabetes by administering a pharmaceutical composition as described herein to subject in need thereof.

The present invention further provides the use of a peptide, peptidomimetic or conjugate as described herein, for the preparation of a medicament for the treatment of Type 2 diabetes.

In some embodiments, there is provided herein a method for treating Type 2 diabetes in a subject in need thereof, the method comprising administering to the subject a pharmaceutical composition comprising a peptide or a peptidomimetic of 7-20 amino acids, the peptide or peptidomimetic comprising the sequence X₁-X₂-X₃-X₄-Lys-Gly-Gln-X₅-Thr-X₆-X₇ (SEQ ID NO: 40), wherein:

X₁ is any amino acid residue;

X₂ is absent or represents a stretch of two amino acid residues selected from the group consisting of: Arg-X₈, wherein X₈ is any amino acid residue, Asp-Met and Leu-Gln;

X₃ is selected from the group consisting of Phe, Tyr, His, Leu, Ala, Ser and NorVal;

X₄ is selected from Thr and Gln;

X₅ is selected from Val and Ser;

X₆ is absent or selected from the group consisting of a positively charged amino acid residue, Ser and Val; and

X₇ is absent or represents a positively charged amino acid residue, optionally modified with a label. In some embodiments, the label is a detectable label.

In some embodiments, the method comprises administering a pharmaceutical composition comprising a peptide or peptidomimetic comprising a sequence selected from the group consisting of:

(SEQ ID NO: 2)
X₁-Arg-Asn-Phe-Gln-Lys-Gly-Gln-Val-Thr-Arg;
(SEQ ID NO: 3)
X₁-Asp-Met-Phe-Thr-Lys-Gly-Gln-Val-Thr-Arg;
(SEQ ID NO: 4)
X₁-Leu-Gln-Ser-Thr-Lys-Gly-Gln-Ser-Thr-Ser;
(SEQ ID NO: 5)
X₁-Leu-Gln-Ser-Gln-Lys-Gly-Gln-Ser-Thr-Ser;
and
(SEQ ID NO: 6)
X₁-Arg-Gly-Phe-Gln-Lys-Gly-Gln-Val-Thr-Val,

wherein X₁ is any amino acid residue.

In some embodiments, the method comprises administering a pharmaceutical composition comprising a peptide or peptidomimetic comprising the sequence Gly-X₂-X₃-X₄-Lys-Gly-Gln-Val-Thr-X₆-X₇ (SEQ ID NO: 8), wherein:

X₂ is absent or represents Arg-Asn;

X₃ is selected from the group consisting of Phe, Tyr, His, Leu, Ala, and NorVal;

X₄ is Thr or Gln;

X₆ is absent or represents a positively charged amino acid residue; and

X₇ is absent or represents a positively charged amino acid residue, optionally modified with a label.

In some embodiments, there is provided herein a method for treating Type 2 diabetes in a subject in need thereof, the method comprising administering to the subject a pharmaceutical composition comprising a synthetic peptide or a peptidomimetic of 7-20 amino acids, the peptide comprising the sequence X₁-X₂-X₃-X₄-Lys-Gly-Gln-Val-Thr-X₆-X₇ (SEQ ID NO: 7), wherein:

X₁ is any amino acid residue;

X₂ is absent or represents Arg-Asn;

X₃ is selected from the group consisting of Phe, Tyr, His, Leu, Ala and NorVal;

X₄ is Thr or Gln;

X₆ is absent or represents a positively charged amino acid residue, e.g., Arg; and

X₇ is absent or represents an amino acid residue, optionally modified with a label, e.g., a positively charged amino acid residue, optionally modified with a label.

According to yet another aspect, the present invention provides a pharmaceutical composition for use in the treatment of Type 2 diabetes in a subject in need thereof, the pharmaceutical composition comprising a synthetic peptide or a peptidomimetic of 7-20 amino acids, the peptide comprising the sequence X₁-X₂-X₃-X₄-Lys-Gly-Gln-Val-Thr-X₆-X₇ (SEQ ID NO: 7), wherein:

X₁ is any amino acid residue;

X₂ is absent or represents Arg-Asn;

X₃ is selected from the group consisting of Phe, Tyr, His, Leu, Ala and NorVal;

X₄ is Thr or Gln;

X₆ is absent or represents a positively charged amino acid residue, e.g., Arg; and

X₇ is absent or represents an amino acid residue, optionally modified with a label, e.g., a positively charged amino acid residue, optionally modified with a label.

In some embodiments, the present invention provides a method for treating Type 2 diabetes in a subject in need thereof, the method comprising administering to the subject a pharmaceutical composition comprising a peptide or a peptidomimetic of 6-26 amino acids comprising the sequence Xₐ-X_{b}-X_{c}-X_{d}-Gly-Gln-Val-Xₑ (SEQ ID NO: 58), wherein:

Xₐ and Xₑ are each independently a stretch of 0-10 amino acid residues;

X_{b} is selected from the group consisting of Phe, Tyr, His, Leu and NorVal;

X_{c} is Thr or Gln; and

X_{d} is selected from the group consisting of Lys, D-Lys, Ac-Lys, Arg and Orn.

In some embodiments, Xₐ and Xₑ are each independently a stretch of 0-5 amino acid residues. In additional embodiments, Xₐ and Xₑ are each independently a stretch of 1-3 amino acid residues.

In some embodiments, Xₐ comprises Gly. In additional embodiments, Xₐ comprises Arg-Asn.

In some embodiments, Xₐ is selected from the group consisting of Gly, Gly-Arg-Asn, Gly-Gln-Phe-Nle-Arg-Asn (SEQ ID NO: 59), and Gly-Nle.

In some embodiments, X_{b} is Phe or Tyr. In additional embodiments, X_{b} is selected from the group consisting of His, Leu and NorVal.

In some embodiments, X_{b}-X_{c}-X_{d} represent Phe-Thr-Lys. In additional embodiments, X_{b}-X_{c}-X_{d} represent Phe-Gln-Lys. In additional embodiments, X_{b}-X_{c}-X_{d} represent His-Gln-Lys. In yet additional embodiments, X_{b}-X_{c}-X_{d} represent Leu-Gln-Lys. In yet additional embodiments, X_{b}-X_{c}-X_{d} represent NorVal-Gln-Lys.

In some embodiments, Xₑ comprises Thr.

In some embodiments, Xₑ is selected from the group consisting of Thr, Thr-Thr, Thr-Arg, Thr-X₇ and Thr-Arg-X₇, wherein X₇ is an amino acid residue modified with a detectable label.

In some embodiments, the method comprises administering a peptide conjugate according to the following formula: R₁-Xₐ-X_{b}-X_{c}-X_{d}-Gly-Gln-Val-Xₑ-R₂, wherein R₁ is selected from the group consisting of a permeability-enhancing moiety and a detectable moiety, linked via a direct bond or via a linker; R₂ designates OH of an unmodified carboxy terminal group or a modified carboxy terminal group; and Xₐ-Xₑ are as defined above.

In some embodiments, the peptide is selected from the group consisting of:

R₁-X_{a′}-Arg-Asn-Phe-Gln-Lys-Gly-Gln-Val-Thr-Arg-Xₑ-R₂; and (i)

R₁-X_{a′}-Phe-Thr-Lys-Gly-Gln-Val-Thr-X_{e′}-R₂, (ii)

wherein:
X_{a′} and X_{e′} are each independently a stretch of 0-8 amino acid residues;
X_{a″} is a stretch of 0-10 amino acid residues;
X_{e′} is a stretch of 0-9 amino acid residues; and
R₁ and R₂ are as defined above.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### Examples

### Example 1—Synthesis of Peptides

A set of peptides derived from a segment of human HO-1 (h-HO-1) or from a similar sequence found in human HIF1α (h-HIF1-α) was designed.

The parent sequences are as follows:

h-HO-1 32-50:
(SEQ ID NO: 60)
EFMRNFQKGQVTRDGFKLV K = Lysine # 39
h-HIF1α 292-308:
(SEQ ID NO: 61)
THHDMFTKGQVTTGQYRML K = Lysine # 297

The complete sequence of h-HO-1 and h-HIF1α are disclosed in UniProt Accession Nos. P09601.1 GI: 123446 and Q16665.1 GI: 2498017, respectively.

The homologous sequences are underlined. The lysine residue (K) marked in boldface indicates the position in HO-1 which was found to undergo reversible post-translational acetylation.

The h-HO-1-derived and h-HIF1α-derived peptides are listed below in Table 1. The sequence corresponding to the native polypeptide sequence in each parent protein is underlined. The position of the Lysine residue (K) that undergoes modification in the native HO-1 is marked in boldface.

The peptides were synthesized using solid phase synthesis and HPLC purified (>95%).

TABLE 1
Peptides

| SEQ | | | |
|---|---|---|---|
| ID | Peptide | | Derived |
| NO. | name | Sequence | from |
| 28 | ACD-004 | Myr-G-R-N-F-Q-K-G-Q-V-T-R-NH₂ | h-HO-1 |
| 39 | ACD-005 | Myr-G-F-T-K-G-Q-V-T-NH₂ | h-HIF1α |
| 31 | ACD-403.3 | Stear-G-R-N-H-Q-K-G-Q-V-T-R-NH₂ | h-HO-1 |
| 32 | ACD-047.7 | Myr-G-R-N-L-Q-K-G-Q-V-T-R-NH₂ | h-HO-1 |
| 33 | ACD-040.2 | Myr-G-R-N-NorVal-Q-K-G-Q-V-T-R-NH₂ | h-HO-1 |
| 34 | ACD-013 | Myr-G-R-N-F-Q-K-G-Q-V-T-R-K(Biotin)-NH₂ | h-HO-1 |
| 30 | ACD-403.1 | Palm-G-R-N-H-Q-K-G-Q-V-T-R-NH₂ | h-HO-1 |
| 35 | ACD-040.1 | Myr-G-R-N-A-Q-K-G-Q-V-T-R-NH₂ | h-HO-1 |
| 36 | ACD-046 | Palm-G-R-N-F-Q-K-G-Q-V-T-R-NH₂ | h-HO-1 |
| 29 | ACD-400.3 | Stear-G-R-N-F-Q-K-G-Q-V-T-R-NH₂ | h-HO-1 |
| 37 | ACD-047.1 | Stear-G-R-N-Y-Q-K-G-Q-V-T-R-NH₂ | h-HO-1 |
| 38 | ACD- | Stear-G-R-N-Y-Q-K-G-Q-V-T-R-K(Dansyl)-NH₂ | h-HO-1 |
| | 047.1* | | |

For all peptides Myr = myristoyl and NH₂ designated C-terminal amidation. Stear = Stearoyl, Palm-Palmitoyl

A sequence similar to the sequence around the acetylation site in the human HO-1 was also identified in the protein DQX1:

h-HO-1 acetylation site:
EFMRNFQKGQVTRDGFKLV K = Lysine # 39
h-HIF1α similar sequence:
THHDMFTKGQVTTGQYRML K = Lysine # 297
h-DQX1 similar seq.
EFALARGFQKGQVTVTQPYPA K = Lysine # 95

(The above sequences are set forth as SEQ ID NOs: 60-62, respectively).

The complete sequence of h-DQX1 is disclosed in UniProt Accession No. Q8TE96 DQX1 is a gene whose epigenetic control is changed significantly between type-2 diabetics and non-diabetics (see: Al Muftah et al., 2016, Clinical Epigenetics, 8:13).

The homologous sequences are underlined. K marked in boldface indicates a putatively acetylated lysine residue.

Table 1A shows an alignment of the homologous sequences around the acetylation site in HO-1.

TABLE 1A
homologous sequences

| Peptide | SEQ ID | | | | | | | | | | |
|---|---|---|---|---|---|---|---|---|---|---|---|
| name | NO: | Sequence | | | | | | | | | |
| HO-1 | 63 | R | N | F | Q | K | G | Q | V | T | R |
| HIF-1 alpha | 64 | D | M | F | T | K | G | Q | V | T | T |
| DQX1 | 65 | R | G | F | Q | K | G | Q | V | T | V |
| Consensus | 66 | R | F | Q | K | G | Q | V | T | | |

K marked in boldface indicates a putatively acetylated lysine residue.

### Example 2—Inhibition of Starvation-Induced Insulin Resistance by ACD-004 and ACD-005

Insulin resistance is developed when normal subjects are exposed to acute starvation (see for example Newman and Brodows, 1982, Metabolism 32:590-6; Bjorkman and Eriksson, 1985, J. Clin. Invest. 76:87-92). The ability of the peptides ACD-004 and ACD-005 described in Example 1 above to inhibit insulin-resistance induced by starvation was tested in mice (male C57BL mice ˜9-10 wks old).

Protocol: Blood glucose was measured in the morning (time 0). The mice were then injected intraperitoneally with a peptide (1 mg/mouse=˜35 mg/kg) or with a vehicle and placed in a cage with water but no food. After 8 hrs starvation, blood glucose was measured again and the mice were supplied with food.

Peptides' solutions for injection were prepared as follows: 10 mg peptide were dissolved in 0.2 ml DMSO. 0.8 ml of 1% Brij®-97 (Sigma-Aldrich) was then added and mixed to homogeneity. Finally, 1 ml of DDW was added and the solution was mixed. 0.2 ml of this solution, or from a vehicle (no peptide), were injected i.p. into the mice.

The results, summarized in Table 2 below, are expressed as % decrease in blood glucose after 8 hrs starvation, compared with blood glucose at time 0. A higher decrease in glucose levels in the blood indicates better intake of glucose by cells, which reflects better inhibition of the starvation-induced insulin resistance.

TABLE 2
Inhibition of fasting insulin-resistance by ACD peptides

| | Core | # | % blood glucose decrease |
|---|---|---|---|
| Treatment | sequence | of mice | following 8 h starvation |
| Vehicle | — | 4 | 14% |
| ACD-004 | --Q-K-G-- | 4 | 47% |
| ACD-005 | --T-K-G-- | 4 | 49% |

As can be seen in the table, ACD-004 and ACD-005 effectively inhibited insulin-resistance that developed in the course of starvation, resulting in a significantly higher decrease in blood glucose level following starvation, compared to vehicle alone.

### Example 3—Db/Db Mice Response to ACD-004 Treatment

ACD-004 was tested for its effect on hyperglycemia in obese diabetic mice (db/db).

Protocol: Mice (n=3) were kept on normal, unlimited diet. Prior to peptide treatment, baseline measurements of blood glucose and body weight were taken every ˜3 weeks through a period of 2.5 months (see time points (−1)-(−4) in Table 3 below). Injections of the peptide (intraperitoneally, ˜35 mg/kg) started on time point 0 and continued on time points 1 and 2, twice a day. Blood glucose and body weight were measured at the indicated days.

The results, summarized in Table 3, are expressed as mean % change in blood glucose compared to the first baseline measurement (time point (−4)).

TABLE 3
Response of (db/db) mice to ACD-004 treatment (mean values):

| Time | Age | Body | Blood glucose | % change from |
|---|---|---|---|---|
| point | (wks) | Wt. (g) | (mg/dL) | time point (−4) |
| (−4) | 7 | 34 | 241 | 0% |
| (−3) | 10.3 | 47.7 | 487 | +102% |
| (−2) | 13.7 | 54.3 | 354 | +47% |
| (−1) | 17.3 | 58.3 | 351 | +46% |
| 0 | 17.7 | 56 | — | — |
| 1 | 17.9 | 55.3 | 177 | −27% |
| 2 | 18.1 | 54 | 139 | −42% |
| 3 | 18.3 | 54 | 105 | −56% |

As can be seen in the table, ACD-004 effectively reduced blood glucose levels in the diabetic mice.

A further experiment with another group of (db/db) mice in which the peptide was injected only once a day showed similar results.

### Example 4—Inhibition of Starvation-Induced Insulin Resistance by ACD-403.3, ACD-047.7 and ACD-040.2

The ability of the peptides ACD-403.3, ACD-047.7 and ACD-040.2 described in Example 1 above, to inhibit insulin-resistance induced by starvation was tested in mice (male C57BL mice ˜9-10 wks old).

Protocol: Blood glucose was measured in the morning (time 0). The mice were then injected intraperitoneally with a peptide (1 mg/mouse=˜35 mg/kg) or with a vehicle and placed in a cage with water but no food. After 8 hrs starvation, blood glucose was measured again and the mice were supplied with food.

Peptides' solutions for injection were prepared as follows: 5 mg peptide were dissolved in 1 ml of a solution composed of 5% hydroxypropyl-beta-cyclodextrin (HPβCD)+2% Propylene glycol (PG)+2% Tween-80 in DDW (Wt/Vol). If needed, the solution was warmed up to 80° C. to facilitate peptide solubilization. 0.2 ml of this solution, or from a vehicle (no peptide), were injected i.p. into the mice.

The results, summarized in Table 4 below, are expressed as % change in blood glucose level after 8 h starvation, compared to t0 (=100%), as well as % decrease in blood glucose after 8 hrs starvation, compared with blood glucose at time 0. A higher decrease in glucose levels in the blood indicates better intake of glucose by cells, which reflects better inhibition of the starvation-induced insulin resistance.

TABLE 4
Inhibition of fasting insulin-resistance by ACD peptides

| | | Mean % change | | % blood glucose |
|---|---|---|---|---|
| | | in blood | +/− | decrease following |
| Treatment | n | glucose 0→8 h* | SEM | 8 h starvation |
| Vehicle | 10 | 90.3 | 5.2 | 9.7% |
| ACD-403.3 | 9 | 63.3** | 2.4 | 36.7% |
| ACD-047.7 | 10 | 60.8** | 4.0 | 39.2% |
| ACD-040.2 | 9 | 63.4** | 6.0 | 36.6% |

*% change in blood glucose level after 8 h starvation compared to t0 (=100%)
**p < 0.002 by Student t-test, compared to Vehicle

As can be seen in the table, ACD-403.3, ACD-047.7 and ACD-040.2 effectively inhibited insulin-resistance that developed in the course of starvation, resulting in a significantly higher decrease in blood glucose level following starvation, compared to vehicle alone.

### Example 5—Inhibition of Starvation-Induced Insulin Resistance by ACD-047.7 Compared to Inactive Peptides with a Similar Structure

The following peptides were tested according to the protocol described in Example 4:

ACD-047.7:
(SEQ ID NO: 31)
Myr-G-R-N-L-Q-K-G-Q-V-T-R-NH₂
ACD-047.8:
(SEQ ID NO: 67)
Myr-G-R-N-I-Q-K-G-Q-V-T-R-NH₂
ACD-047.9:
(SEQ ID NO: 68)
Myr-G-R-N-Nle-Q-K-G-Q-V-T-R-NH₂

In this experiment, mice were injected with the designated peptide at a dose of 20 mg/kg (i.p).

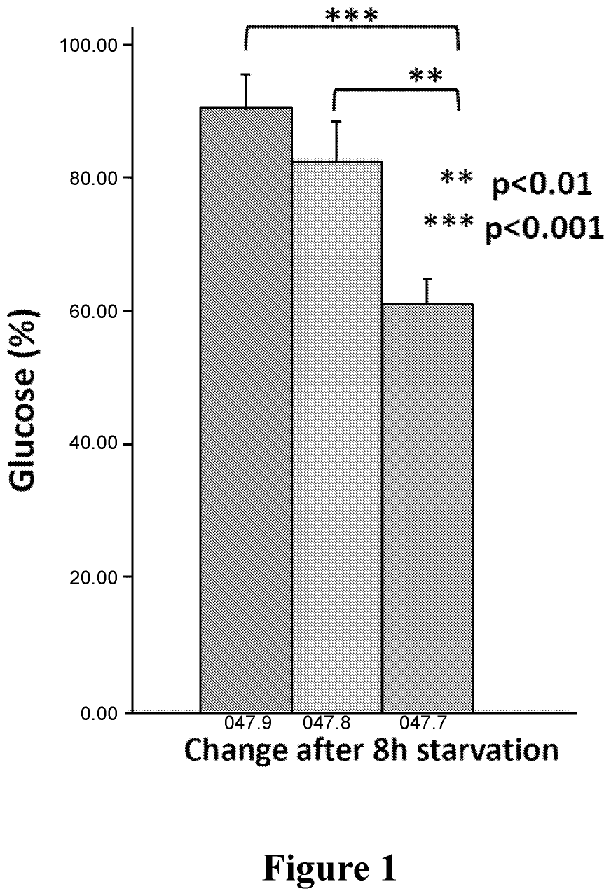

The results, expressed as the relative change in blood glucose level compared to time 0′ (=100%), are summarized in FIG. 1. While peptide ACD-047.7 inhibited significantly the starvation-induced insulin-resistance, two other peptides with a very similar sequence, ACD-047.8 and ACD-047.9, were inactive.

### Example 6—Response of (Db/Db) Mice to Treatment with ACD-400.3

Peptide ACD-400.3 is identical to peptide ACD-004 except that the acyl group in its N-terminus is stearoyl instead of myristoyl:

ACD-400.3:
(SEQ ID NO: 29)
Stear-G-R-N-F-Q-K-G-Q-V-T-R-NH₂

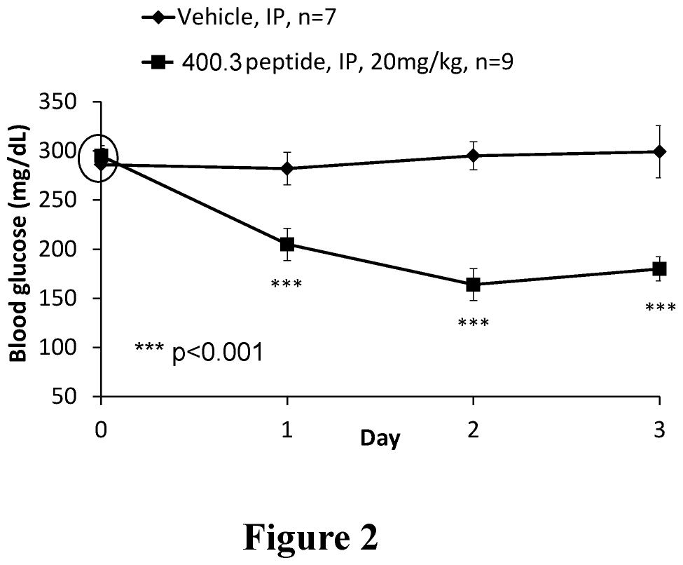

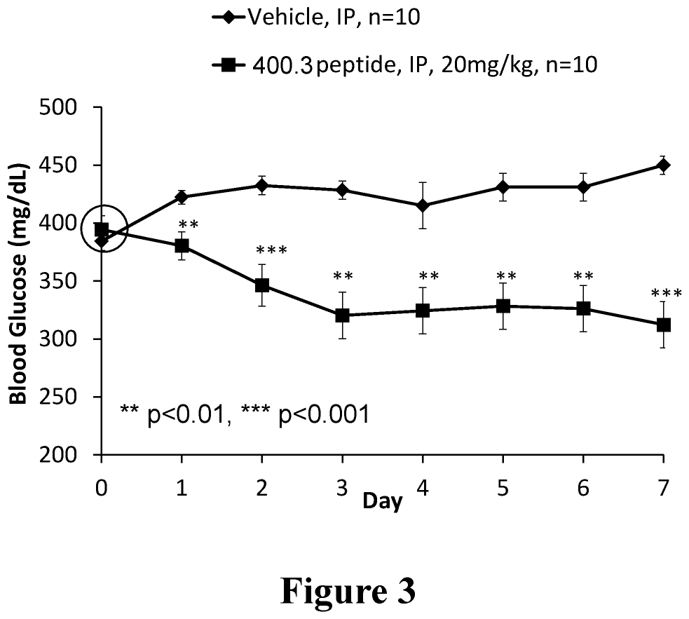

Mice (n=16) were kept on normal, unlimited diet. Injections of the peptide (i.p., 20 mg/kg, n=9) or vehicle (i.p., n=7) started at 3-4 m of age (time point ‘0’) and continued for 3 days, once a day. Blood glucose was measured daily and the results are summarized in FIG. 2. In a similar experiment the peptide or mice were injected to mice with severe diabetes for 7 days. The results are summarized in FIG. 3.

As can be seen, there is an outstanding decrease in blood glucose following treatment with the peptide.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1-45. (canceled)

46. A peptide or peptidomimetic of 8-20 amino acids, the peptide or peptidomimetic comprising the sequence X₁-X₂-X₃-X₄-Lys-Gly-Gln-X₅-Thr-X₆-X₇ (SEQ ID NO: 1), wherein:
X₁ is an amino acid residue other than Met and His;
X₂ is absent or represents a stretch of two amino acid residues selected from the group consisting of: Arg-X₈, wherein X₈ is any amino acid residue, Asp-Met and Leu-Gln;
X₃ is selected from the group consisting of Phe, Tyr, His, Leu, Ala, Ser and NorVal;
X₄ is selected from Thr and Gln;
X₅ is selected from Val and Ser;
X₆ is a positively charged amino acid residue; and
X₇ is absent, or represents a positively charged amino acid residue, optionally conjugated with a label.

47. The peptide or peptidomimetic of claim 46, comprising a sequence selected from the group consisting of:
(SEQ ID NO: 2)
X₁-Arg-Asn-Phe-Gln-Lys-Gly-Gln-Val-Thr-Arg,
and
(SEQ ID NO: 3)
X₁-Asp-Met-Phe-Thr-Lys-Gly-Gln-Val-Thr-Arg,
wherein X₁ is as defined in claim 46.

48. The peptide or peptidomimetic of claim 46, comprising a sequence selected from the group consisting of X₁-X₂-X₃-X₄-Lys-Gly-Gln-Val-Thr-X₆-X₇ (SEQ ID NO: 7) and Gly-X₂-X₃-X₄-Lys-Gly-Gln-Val-Thr-X₆-X₇ (SEQ ID NO: 8), wherein:
X₁ is an amino acid residue other than Met;
X₂ is absent or represents Arg-Asn;
X₃ is selected from the group consisting of Phe, Tyr, His, Leu, Ala, and NorVal;
X₄ is Thr or Gln;
X₆ represents a positively charged amino acid residue; and
X₇ is absent or represents a positively charged amino acid residue, optionally conjugated with a label.

49. The peptide or peptidomimetic of claim 48, wherein X₁-X₂-X₃-X₄ represent a stretch of amino acid residues selected from the group consisting of:
(SEQ ID NO: 9)
Gly-Arg-Asn-Phe-Gln;
(SEQ ID NO: 10)
Gly-Arg-Asn-His-Gln;
(SEQ ID NO: 11)
Gly-Arg-Asn-Leu-Gln;
(SEQ ID NO: 12)
Gly-Arg-Asn-NorVal-Gln;
(SEQ ID NO: 13)
Gly-Arg-Asn-Ala-Gln;
(SEQ ID NO: 14)
Gly-Arg-Asn-Tyr-Gln;
and
(SEQ ID NO: 15)
Gly-Phe-Thr.

50. The peptide or peptidomimetic of claim 48, wherein X₆-X₇ represent Arg or Arg-Lys(Z), wherein Z is a detectable label connected to the epsilon amino group of the Lys residue.

51. The peptide or peptidomimetic of claim 48, comprising a sequence selected from X₁-Arg-Asn-X₃-Gln-Lys-Gly-Gln-Val-Thr-Arg-X₇ (SEQ ID NO: 16) and Gly-Arg-Asn-X₃-Gln-Lys-Gly-Gln-Val-Thr-Arg-X₇ (SEQ ID NO: 17), wherein X₁, X₃ and X₇ are as defined in claim 48.

52. The peptide or peptidomimetic of claim 51, comprising a sequence selected from the group consisting of:
(SEQ ID NO: 18)
Gly-Arg-Asn-Phe-Gln-Lys-Gly-Gln-Val-Thr-Arg;
(SEQ ID NO: 19)
Gly-Arg-Asn-His-Gln-Lys-Gly-Gln-Val-Thr-Arg;
(SEQ ID NO: 20)
Gly-Arg-Asn-Leu-Gln-Lys-Gly-Gln-Val-Thr-Arg;
(SEQ ID NO: 21)
Gly-Arg-Asn-NorVal-Gln-Lys-Gly-Gln-Val-Thr-Arg;
(SEQ ID NO: 22)
Gly-Arg-Asn-Phe-Gln-Lys-Gly-Gln-Val-Thr-Arg-Lys(Z);
(SEQ ID NO: 23)
Gly-Arg-Asn-Ala-Gln-Lys-Gly-Gln-Val-Thr-Arg;
(SEQ ID NO: 24)
Gly-Arg-Asn-Tyr-Gln-Lys-Gly-Gln-Val-Thr-Arg;
and
(SEQ ID NO: 25)
Gly-Arg-Asn-Tyr-Gln-Lys-Gly-Gln-Val-Thr-Arg-Lys(Z),
wherein Z is as defined in claim 50.

53. The peptide or peptidomimetic of claim 46, wherein the amino terminus of the synthetic peptide or peptidomimetic is modified with an amino-terminal blocking group selected from the group consisting of an acyl, alkyl and aryl.

54. The peptide or peptidomimetic of claim 46, wherein the carboxy terminus of the synthetic peptide or peptidomimetic is modified with a group selected from amide, ester and alcohol group.

55. A conjugate comprising a peptide or peptidomimetic according to claim 46 and at least one moiety selected from the group consisting of a permeability-enhancing moiety, a detectable label and a carrier.

56. The conjugate of claim 55, wherein the conjugate is according to Formula I: R₁-X₁-X₂-X₃-X₄-Lys-Gly-Gln-X₅-Thr-X₆-X₇-R₂, wherein R₁ is selected from the group consisting of a permeability-enhancing moiety and a detectable label, linked via a direct bond or via a linker; R₂ designates OH of an unmodified carboxy terminal group or a modified carboxy terminal group; and X₁-X₇ are as defined in claim 46.

57. The conjugate of claim 56, wherein the conjugate is according to Formula Ia:
R₁-X₁-Arg-Asn-X₃-Gln-Lys-Gly-Gln-Val-Thr-Arg-X₇-R₂,
wherein R₁ and R₂ are as defined in claim 56, and wherein:
X₁ is an amino acid residue other than Met;
X₃ is selected from the group consisting of Phe, Tyr, His, Leu, Ala, and NorVal; and
X₇ is absent or represents a positively charged amino acid residue, optionally conjugated with a label.

58. The conjugate of claim 57, wherein the conjugate is according to a formula selected from the group consisting of:
R₁-Gly-Arg-Asn-Phe-Gln-Lys-Gly-Gln-Val-Thr-Arg-R₂;
R₁-Gly-Arg-Asn-His-Gln-Lys-Gly-Gln-Val-Thr-Arg-R₂;
R₁-Gly-Arg-Asn-Leu-Gln-Lys-Gly-Gln-Val-Thr-Arg-R₂;
R₁-Gly-Arg-Asn-NorVal-Gln-Lys-Gly-Gln-Val-Thr-Arg-R₂;
R₁-Gly-Arg-Asn-Phe-Gln-Lys-Gly-Gln-Val-Thr-Arg-Lys(Z)-R₂;
R₁-Gly-Arg-Asn-Ala-Gln-Lys-Gly-Gln-Val-Thr-Arg-R₂;
R₁-Gly-Arg-Asn-Tyr-Gln-Lys-Gly-Gln-Val-Thr-Arg-R₂; and
R₁-Gly-Arg-Asn-Tyr-Gln-Lys-Gly-Gln-Val-Thr-Arg-Lys(Z)-R₂,
wherein Z is a detectable label and R₁, R₂ are as defined in claim 56.

59. The conjugate of claim 58, wherein the conjugate is selected from the group consisting of:
(SEQ ID NO: 28)
Myr-Gly-Arg-Asn-Phe-Gln-Lys-Gly-Gln-Val-Thr-Arg-
NH₂,;
(SEQ ID NO: 29)
Stear-Gly-Arg-Asn-Phe-Gln-Lys-Gly-Gln-Val-Thr-Arg-
NH₂,;
(SEQ ID NO: 30)
Palm-Gly-Arg-Asn-His-Gln-Lys-Gly-Gln-Val-Thr-Arg-
NH₂;
(SEQ ID NO: 31)
Stear-Gly-Arg-Asn-His-Gln-Lys-Gly-Gln-Val-Thr-Arg-
NH₂;
(SEQ ID NO: 32)
Myr-Gly-Arg-Asn-Leu-Gln-Lys-Gly-Gln-Val-Thr-Arg-
NH₂;
(SEQ ID NO: 33)
Myr-Gly-Arg-Asn-NorVal-Gln-Lys-Gly-Gln-Val-Thr-
Arg-NH₂;
(SEQ ID NO: 34)
Myr-Gly-Arg-Asn-Phe-Gln-Lys-Gly-Gln-Val-Thr-Arg-
Lys(Biotin)-NH₂;
(SEQ ID NO: 35)
Myr-Gly-Arg-Asn-Ala-Gln-Lys-Gly-Gln-Val-Thr-Arg-
NH₂;
(SEQ ID NO: 36)
Palm-Gly-Arg-Asn-Phe-Gln-Lys-Gly-Gln-Val-Thr-Arg-
NH₂,;
(SEQ ID NO: 37)
Stear-Gly-Arg-Asn-Tyr-Gln-Lys-Gly-Gln-Val-Thr-Arg-
NH₂;
and
(SEQ ID NO: 38)
Stear-Gly-Arg-Asn-Tyr-Gln-Lys-Gly-Gln-Val-Thr-Arg-
Lys(Dansyl)-NH₂.

60. A pharmaceutical composition comprising as an active ingredient a peptide or peptidomimetic according to claim 46 or a conjugate thereof, and a pharmaceutically acceptable carrier.

61. A method for treating Type 2 diabetes in a subject in need thereof, the method comprising administering a pharmaceutical composition according to claim 60 to said subject.

62. A peptide or peptidomimetic of 8-20 amino acids, the peptide or peptidomimetic comprising the sequence X₁-Phe-Thr-Lys-Gly-Gln-Val-Thr (SEQ ID NO: 26), wherein X₁ is an amino acid residue other than Met.

63. The peptide or peptidomimetic of claim 62, comprising the sequence Gly-Phe-Thr-Lys-Gly-Gln-Val-Thr (SEQ ID NO: 27).

64. A conjugate comprising a peptide or peptidomimetic according to claim 62 and at least one moiety selected from the group consisting of a permeability-enhancing moiety, a detectable label and a carrier.

65. The conjugate of claim 64, wherein the conjugate is Myr-Gly-Phe-Thr-Lys-Gly-Gln-Val-Thr-NH₂ (SEQ ID NO: 39).

66. A method for treating Type 2 diabetes in a subject in need thereof, the method comprising administering to the subject a pharmaceutical composition comprising a peptide or a peptidomimetic of 7-20 amino acids, the peptide or peptidomimetic comprising the sequence X₁-X₂-X₃-X₄-Lys-Gly-Gln-X₅-Thr-X₆-X₇ (SEQ ID NO: 40), wherein:
X₁ is any amino acid residue;
X₂ is absent or represents a stretch of two amino acid residues selected from the group consisting of: Arg-X₈, wherein X₈ is any amino acid residue, Asp-Met and Leu-Gln;
X₃ is selected from the group consisting of Phe, Tyr, His, Leu, Ala, Ser and NorVal;
X₄ is selected from Thr and Gln;
X₅ is selected from Val and Ser;
X₆ is absent or selected from the group consisting of a positively charged amino acid residue, Ser and Val; and
X₇ is absent, or represents a positively charged amino acid residue, optionally conjugated with a label.


SEQUENCE LISTING (68 sequences)

SEQ ID NO: 1 - protein, 11 aa, Artificial Sequence
  Peptide
  MISC_FEATURE (1)..(1): X1 = an amino acid residue other than Met and His
  MISC_FEATURE (2)..(2): X2 = absent or represents a stretch of two amino acid residues selected from the group consisting of Arg-X8, wherein X8 is any amino acid residue, Asp-Met and Leu-Gln
  MISC_FEATURE (3)..(3): X3 = Phe, Tyr, His, Leu, Ala, Ser or Nva
  MISC_FEATURE (4)..(4): X4 = Thr or Gln
  MISC_FEATURE (8)..(8): X5 =  Val or Ser
  MISC_FEATURE (10)..(10): X6 = absent or selected from a positively charged amino acid residue, Ser and Val
  MISC_FEATURE (11)..(11): X7 = absent or represents a positively charged amino acid residue, optionally conjugated with a label
XXXXKGQXTXX

SEQ ID NO: 2 - protein, 11 aa, Artificial Sequence
  Peptide
  MISC_FEATURE (1)..(1): X= an amino acid residue other than Met and His
XRNFQKGQVTR

SEQ ID NO: 3 - protein, 11 aa, Artificial Sequence
  Peptide
  MISC_FEATURE (1)..(1): X= an amino acid residue other than Met and His
XDMFTKGQVTR

SEQ ID NO: 4 - protein, 11 aa, Artificial Sequence
  Peptide
  MISC_FEATURE (1)..(1): X= an amino acid residue other than Met and His
XLQSTKGQSTS

SEQ ID NO: 5 - protein, 11 aa, Artificial Sequence
  Peptide
  MISC_FEATURE (1)..(1): X= an amino acid residue other than Met and His
XLQSQKGQSTS

SEQ ID NO: 6 - protein, 11 aa, Artificial Sequence
  Peptide
  MISC_FEATURE (1)..(1): X=an amino acid residue other than Met and His
XRGFQKGQVTV

SEQ ID NO: 7 - protein, 11 aa, Artificial Sequence
  Peptide
  MISC_FEATURE (1)..(1): X = an amino acid residue other than Met
  MISC_FEATURE (2)..(2): X = absent or represents Arg-Asn
  MISC_FEATURE (3)..(3): X = Phe, Tyr, His, Leu, Ala or Nva
  MISC_FEATURE (4)..(4): X = Thr or Gln
  MISC_FEATURE (10)..(10): X = absent or represents a positively charged amino acid residue
  MISC_FEATURE (11)..(11): X = absent or represents a positively charged amino acid residue, optionally conjugated with a label
XXXXKGQVTXX

SEQ ID NO: 8 - protein, 11 aa, Artificial Sequence
  Peptide
  MISC_FEATURE (2)..(2): X= absent or represents Arg-Asn
  MISC_FEATURE (3)..(3): X= Phe, Tyr, His, Leu, Ala or Nva
  MISC_FEATURE (4)..(4): X= Thr or Gln
  MISC_FEATURE (10)..(10): X= absent or represents a positively charged amino acid residue
  MISC_FEATURE (11)..(11): X= absent or represents a positively charged amino acid residue, optionally conjugated with a label
GXXXKGQVTXX

SEQ ID NO: 9 - protein, 5 aa, Artificial Sequence
  Peptide
GRNFQ

SEQ ID NO: 10 - protein, 5 aa, Artificial Sequence
  Peptide
GRNHQ

SEQ ID NO: 11 - protein, 5 aa, Artificial Sequence
  Peptide
GRNLQ

SEQ ID NO: 12 - protein, 5 aa, Artificial Sequence
  Peptide
  MISC_FEATURE (4)..(4): X= Norvaline (Nva)
GRNXQ

SEQ ID NO: 13 - protein, 5 aa, Artificial Sequence
  Peptide
GRNAQ

SEQ ID NO: 14 - protein, 5 aa, Artificial Sequence
  Peptide
GRNYQ

SEQ ID NO: 15 - protein, 3 aa, Artificial Sequence
  Peptide
GFT

SEQ ID NO: 16 - protein, 12 aa, Artificial Sequence
  Peptide
  MISC_FEATURE (1)..(1): X= an amino acid residue other than Met
  MISC_FEATURE (4)..(4): X= Phe, Tyr, His, Leu, Ala or Nva
  MISC_FEATURE (12)..(12): X= absent or represents a positively charged amino acid residue, optionally conjugated with a label
XRNXQKGQVTRX

SEQ ID NO: 17 - protein, 12 aa, Artificial Sequence
  Peptide
  MISC_FEATURE (4)..(4): X= Phe, Tyr, His, Leu, Ala or Nva
  MISC_FEATURE (12)..(12): X= absent or represents a positively charged amino acid residue, optionally modified with a label
GRNXQKGQVTRX

SEQ ID NO: 18 - protein, 11 aa, Artificial Sequence
  Peptide
GRNFQKGQVTR

SEQ ID NO: 19 - protein, 11 aa, Artificial Sequence
  Peptide
GRNHQKGQVTR

SEQ ID NO: 20 - protein, 11 aa, Artificial Sequence
  Peptide
GRNLQKGQVTR

SEQ ID NO: 21 - protein, 11 aa, Artificial Sequence
  Peptide
  MISC_FEATURE (4)..(4): X= Nva
GRNXQKGQVTR

SEQ ID NO: 22 - protein, 12 aa, Artificial Sequence
  Peptide
  MISC_FEATURE (12)..(12): Lys modified with a label
GRNFQKGQVTRK

SEQ ID NO: 23 - protein, 11 aa, Artificial Sequence
  Peptide
GRNAQKGQVTR

SEQ ID NO: 24 - protein, 11 aa, Artificial Sequence
  Peptide
GRNYQKGQVTR

SEQ ID NO: 25 - protein, 12 aa, Artificial Sequence
  Peptide
  MISC_FEATURE (12)..(12): Lys modified with a label
GRNYQKGQVTRK

SEQ ID NO: 26 - protein, 8 aa, Artificial Sequence
  Peptide
  MISC_FEATURE (1)..(1): X= an amino acid residue other than Met
XFTKGQVT

SEQ ID NO: 27 - protein, 8 aa, Artificial Sequence
  Peptide
GFTKGQVT

SEQ ID NO: 28 - protein, 11 aa, Artificial Sequence
  Peptide
  MISC_FEATURE (1)..(1): N-terminal myristoyl group
  MISC_FEATURE (11)..(11): C-terminal amide group
GRNFQKGQVTR

SEQ ID NO: 29 - protein, 11 aa, Artificial Sequence
  Peptide
  MISC_FEATURE (1)..(1): N-terminal stearoyl group
  MISC_FEATURE (11)..(11): C-terminal amide group
GRNFQKGQVTR

SEQ ID NO: 30 - protein, 11 aa, Artificial Sequence
  Peptide
  MISC_FEATURE (1)..(1): N-terminal palmitoyl group
  MISC_FEATURE (11)..(11): C-terminal amide group
GRNHQKGQVTR

SEQ ID NO: 31 - protein, 11 aa, Artificial Sequence
  Peptide
  MISC_FEATURE (1)..(1): N-terminal stearoyl group
  MISC_FEATURE (11)..(11): C-terminal amide group
GRNHQKGQVTR

SEQ ID NO: 32 - protein, 11 aa, Artificial Sequence
  Peptide
  MISC_FEATURE (1)..(1): N-terminal myristoyl group
  MISC_FEATURE (11)..(11): C-terminal amide group
GRNLQKGQVTR

SEQ ID NO: 33 - protein, 11 aa, Artificial Sequence
  Peptide
  MISC_FEATURE (1)..(1): N-terminal myristoyl group
  MISC_FEATURE (4)..(4): X= Nva
  MISC_FEATURE (11)..(11): C-terminal amide group
GRNXQKGQVTR

SEQ ID NO: 34 - protein, 12 aa, Artificial Sequence
  Peptide
  MISC_FEATURE (1)..(1): N-terminal myristoyl
  MISC_FEATURE (12)..(12): Lys modified with biotin; C-terminal amide group
GRNFQKGQVTRK

SEQ ID NO: 35 - protein, 11 aa, Artificial Sequence
  Peptide
  MISC_FEATURE (1)..(1): N-terminal myristoyl group
  MISC_FEATURE (11)..(11): C-terminal amide group
GRNAQKGQVTR

SEQ ID NO: 36 - protein, 11 aa, Artificial Sequence
  Peptide
  MISC_FEATURE (1)..(1): N-terminal palmitoyl group
  MISC_FEATURE (11)..(11): C-terminal amide group
GRNFQKGQVTR

SEQ ID NO: 37 - protein, 11 aa, Artificial Sequence
  Peptide
  MISC_FEATURE (1)..(1): N-terminal stearoyl group
  MISC_FEATURE (11)..(11): C-terminal amide group
GRNYQKGQVTR

SEQ ID NO: 38 - protein, 12 aa, Artificial Sequence
  Peptide
  MISC_FEATURE (1)..(1): N-terminal stearoyl group
  MISC_FEATURE (12)..(12): Lys modified with a dansyl group; C-terminal amide group
GRNYQKGQVTRK

SEQ ID NO: 39 - protein, 8 aa, Artificial Sequence
  Peptide
  MISC_FEATURE (1)..(1): N-terminal myristoyl group
  MISC_FEATURE (8)..(8): C-terminal amide group
GFTKGQVT

SEQ ID NO: 40 - protein, 11 aa, Artificial Sequence
  Peptide
  MISC_FEATURE (1)..(1): X= any amino acid residue
  MISC_FEATURE (2)..(2): X= absent or represents a stretch of two amino acid residues selected from Arg-X8, wherein X8 is any amino acid residue, Asp-Met and Leu-Gln
  MISC_FEATURE (3)..(3): X= Phe, Tyr, His, Leu, Ala, Ser or Nva
  MISC_FEATURE (4)..(4): X= Thr or Gln
  MISC_FEATURE (8)..(8): X= Val or Ser
  MISC_FEATURE (10)..(10): X= absent or selected from a positively charged amino acid residue, Ser and Val
  MISC_FEATURE (11)..(11): X= absent or represents a positively charged amino acid residue, optionally conjugated with a label
XXXXKGQXTXX

SEQ ID NO: 41 - protein, 11 aa, Artificial Sequence
  Peptide
  MISC_FEATURE (1)..(1): X= any amino acid
  MISC_FEATURE (4)..(4): X= Phe, Tyr, His, Leu, Ala or Nva
  MISC_FEATURE (5)..(5): X=Thr or Gln
XRNXXKGQVTR

SEQ ID NO: 42 - protein, 10 aa, Artificial Sequence
  Peptide
  MISC_FEATURE (3)..(3): X= Phe, Tyr, His, Leu, Ala or Nva
  MISC_FEATURE (4)..(4): X= Thr or Gln
RNXXKGQVTR

SEQ ID NO: 43 - protein, 9 aa, Artificial Sequence
  Peptide
  MISC_FEATURE (1)..(1): X= a stretch of 0-10 amino acid residues
  MISC_FEATURE (2)..(2): X= an amino acid residue other than Met
  MISC_FEATURE (3)..(3): X= Phe, Tyr, His, Leu or Nva
  MISC_FEATURE (4)..(4): X= Thr or Gln
  MISC_FEATURE (5)..(5): X= Lys, D-Lys, AcLys, Arg or Orn
  MISC_FEATURE (9)..(9): X= a stretch of 0-10 amino acid residues
XXXXXGQVX

SEQ ID NO: 44 - protein, 5 aa, Artificial Sequence
  Peptide
  MISC_FEATURE (4)..(4): X= Nle
GQFXR

SEQ ID NO: 45 - protein, 11 aa, Artificial Sequence
  Peptide
  MISC_FEATURE (1)..(1): N-terminal myristoy group
  MISC_FEATURE (11)..(11): C-terminal amide group
GRNYQKGQVTR

SEQ ID NO: 46 - protein, 9 aa, Artificial Sequence
  Peptide
  MISC_FEATURE (1)..(1): N-terminal myristoyl group
  MISC_FEATURE (9)..(9): C-terminal amide group
GFQKGQVTR

SEQ ID NO: 47 - protein, 11 aa, Artificial Sequence
  Peptide
  MISC_FEATURE (1)..(1): N-terminal RhodaminB
  MISC_FEATURE (11)..(11): C-terminal amide group
GRNFQKGQVTR

SEQ ID NO: 48 - protein, 14 aa, Artificial Sequence
  Peptide
  MISC_FEATURE (1)..(1): N-terminal myristoyl group
  MISC_FEATURE (4)..(4): X= Nle
  MISC_FEATURE (14)..(14): C-terminal amide group
GQFXRNFQKGQVTR

SEQ ID NO: 49 - protein, 9 aa, Artificial Sequence
  Peptide
  MISC_FEATURE (1)..(1): N-terminal myristoyl group
  MISC_FEATURE (2)..(2): X= Nle
  MISC_FEATURE (9)..(9): C-terminal amide group
GXFTKGQVT

SEQ ID NO: 50 - protein, 9 aa, Artificial Sequence
  Peptide
  MISC_FEATURE (1)..(1): N-terminal myristoyl group
  MISC_FEATURE (9)..(9): C-terminal amide group
GFTKGQVTT

SEQ ID NO: 51 - protein, 9 aa, Artificial Sequence
  Peptide
  MISC_FEATURE (1)..(1): N-terminal myristoyl group
  MISC_FEATURE (9)..(9): Lys modified with biotin; C-terminal amide group
GFTKGQVTK

SEQ ID NO: 52 - protein, 8 aa, Artificial Sequence
  Peptide
  MISC_FEATURE (1)..(1): N-terminal RhodamineB
  MISC_FEATURE (8)..(8): C-terminal amide group
GFTKGQVT

SEQ ID NO: 53 - protein, 11 aa, Artificial Sequence
  Peptide
  MISC_FEATURE (1)..(1): N-terminal myristoyl group
  MISC_FEATURE (11)..(11): C-terminal amide group
GRNFTKGQVTR

SEQ ID NO: 54 - protein, 11 aa, Artificial Sequence
  Peptide
  MISC_FEATURE (1)..(1): N-terminal stearoyl group
  MISC_FEATURE (11)..(11): C-terminal amide group
GRNHQKGQVTR

SEQ ID NO: 55 - protein, 4 aa, Artificial Sequence
  Peptide
  MISC_FEATURE (4)..(4): X= Nle
GQFX

SEQ ID NO: 56 - protein, 7 aa, Artificial Sequence
  Peptide
FTKGQVT

SEQ ID NO: 57 - protein, 10 aa, Artificial Sequence
  Peptide
RNFQKGQVTR

SEQ ID NO: 58 - protein, 8 aa, Artificial Sequence
  Peptide
  MISC_FEATURE (1)..(1): X= a stretch of 0-10 amino acid residues
  MISC_FEATURE (2)..(2): X= Phe, Tyr, His, Leu or Nva
  MISC_FEATURE (3)..(3): X= Thr or Gln
  MISC_FEATURE (4)..(4): X= Lys, D-Lys, AcLys, Arg or Orn
  MISC_FEATURE (8)..(8): X= a stretch of 0-10 amino acid residues
XXXXGQVX

SEQ ID NO: 59 - protein, 6 aa, Artificial Sequence
  Peptide
  MISC_FEATURE (4)..(4): X= Nle
GQFXRN

SEQ ID NO: 60 - protein, 19 aa, Homo sapiens
EFMRNFQKGQVTRDGFKLV

SEQ ID NO: 61 - protein, 19 aa, Homo sapiens
THHDMFTKGQVTTGQYRML

SEQ ID NO: 62 - protein, 21 aa, Homo sapiens
EFALARGFQKGQVTVTQPYPA

SEQ ID NO: 63 - protein, 10 aa, Homo sapiens
RNFQKGQVTR

SEQ ID NO: 64 - protein, 10 aa, Homo sapiens
DMFTKGQVTT

SEQ ID NO: 65 - protein, 10 aa, Homo sapiens
RGFQKGQVTV

SEQ ID NO: 66 - protein, 8 aa, Artificial Sequence
  Peptide
RFQKGQVT

SEQ ID NO: 67 - protein, 11 aa, Artificial Sequence
  Peptide
  MISC_FEATURE (1)..(1): N-terminal myristoyl group
  MISC_FEATURE (11)..(11): C-terminal amide group
GRNIQKGQVTR

SEQ ID NO: 68 - protein, 11 aa, Artificial Sequence
  Peptide
  MISC_FEATURE (1)..(1): N-terminal myristoyl group
  MISC_FEATURE (4)..(4): X= Nle
  MISC_FEATURE (11)..(11): C-terminal amide group
GRNXQKGQVTR